SEQUENCE LISTING (6 sequences)

SEQ ID NO: 1 - protein, 123 aa, Synthetic construct
MNQCMSANEAAITDSAVAVAAASSTHRKVASSTTQYDHKESCNGLYYQGSCYILHSDYKSFEDAKANCAAESSTLPNKSDVLTTWLIDYVEDTWGSDGNPITKTTSDYQDSDVSQEVRKYFCT

SEQ ID NO: 2 - protein, 124 aa, Synthetic construct
RLNQCMSANKAAITDSAVAVAAASSTHRKVVSSTTQYDHKESCNGLYYQGSCYILHSDYKSFEDAKANCAAESSTLPNKSDVLTTWLIDYVEDTWGSDGNPITKTTSDYQDSDVSQEVRKYFCT

SEQ ID NO: 3 - protein, 110 aa, Synthetic construct
MDGTLFPGDDDLAIPATEFFSTKADKKPEAKREAIVKADEDDNEETLKQRLTNLEKKITNVTTKFEQIEKCCKRNDEVLFRLENHAETLRAAMISLAKKIDVQTGRRPYE

SEQ ID NO: 4 - protein, 262 aa, Synthetic construct
YSTCTVPTMNNAKLTSTETSFNDKQKVTFTCDSGYHSLDPNAVCETDKWKYENPCKKMCTVSDYVSELYDKPLYEVNSTMTLSCNGETKYFRCEEKNGNTSWNDTVTCPNAECQPLQLEHGSCQPVKEKYSFGEYMTINCDVGYEVIGVSYISCTANSWNVIPSCQQKCDIPSLSNGLISGSTFSIGGVIHLSCKSGFTLTGSPSSTCIDGKWNPILPTCVRSNEEFDPVDDGPDDETDLSKLSKDVVQYEQEIESLEATYH

SEQ ID NO: 5 - protein, 278 aa, Synthetic construct
MAAVKTPVIVVPVIDRPPSETFPNVHEHINDQKFDDVKDNEVMQEKRDVVIVNDDPDHYKDYVFIQWTGGNIRDDDKYTHFFSGFCNTMCTEETKRNIARHLALWDSKFFTELENKNVEYVVIIENDNVIEDITFLRPVLKAIHDKKIDILQMREIITGNKVKTELVIDKDHAIFTYTGGYDVSLSAYIIRVTTALNIVDEIIKSGGLSSGFYFEIARIENEMKINRQIMDNSAKYVEHDPRLVAEHRFETMKPNFWSRIGTVAAKRYPGVMYTFTTP

SEQ ID NO: 6 - protein, 156 aa, Synthetic construct
MASILNTLRFLEKTSFYNCNDSITKEKIKIKHKGMSFVFYKPKHSTVVKYLSGGGIYHDDLVVLGKVTINDLKMMLFYMDLSYHGVTSSGAIYKLGSSIDRLSLNRTIVTKVNNNYNNYNNYNNYNCYNNYNCYNYDDTFFDDDDGGGGSHHHHHH

(19) United States
(12) Patent Application Publication
(10) Pub. No.: US 2025/0147043 A1
(43) Pub. Date: May 8, 2025

(54) METHODS AND SYSTEMS FOR DIAGNOSING ORTHOPOXVIRUS INFECTION

(71) Applicant: Abbott Laboratories, Abbott Park, IL (US)

(72) Inventors: Abbas X. Hadji, Abbott Park, IL (US); Xiaoxing Qiu, Abbott Park, IL (US)

(21) Appl. No.: 19/012,242

(22) Filed: Jan. 7, 2025

Related U.S. Application Data
(63) Continuation of application No. PCT/US24/51179, filed on Oct. 14, 2024.
(60) Provisional application No. 63/590,476, filed on Oct. 16, 2023.

(51) Int. Cl. G01N 33/68; G01N 33/543; G01N 33/577

(52) U.S. Cl. CPC G01N 33/6854; G01N 33/543; G01N 33/577; G01N 2469/20; G01N 2800/26

(57) ABSTRACT

Provided herein are compositions, methods, and systems for diagnosing orthopoxvirus infection in a subject and/or determining orthopoxvirus vaccination status in a subject.

### RELATED APPLICATION INFORMATION

This application is a continuation application of International Application No. PCT/US24/51179, filed on Oct. 14, 2024, which claims priority to U.S. Application No. 63/590,476, filed on Oct. 16, 2023, the contents of which are herein incorporated by reference.

### FIELD

Provided herein are compositions, methods, and systems for diagnosing orthopoxvirus infection in a subject and/or determining orthopoxvirus vaccination status in a subject.

### SEQUENCE LISTING STATEMENT

The contents of the electronic sequence listing titled ABBTL_41770_302_SequenceListing.xml (Size 7.349 bytes; and Date of Creation Jan. 7, 2025) are herein incorporated by reference in their entirety.

### BACKGROUND

Monkeypox virus (MPXV) is a member of the genus Orthopoxvirus (OPXV) and exhibits high protein homology with the other OPXV species including the eradicated variola virus causing smallpox, the vaccinia virus used as smallpox vaccine, cowpox virus, and camelpox virus.

Current state of the art diagnoses of acute OPXV infections largely utilize polymerase chain reaction (PCR)-based methods detecting molecular marker (OPXV DNA) on lesion specimens, which also can differentiate between different OPXV species. Protein-based methods detecting OPXV antigens from lesion samples have also been developed for diagnoses of acute OPXV infections. When no virologic specimen is available, serologic methods are useful for evaluation of exposure and immunity to OPXV. However, monkeypox virus, together with other orthopoxviruses, is a very large and complex virus. Due to such complexity, it is unknown which viral protein elicits the immunodominant response that can be used as a serologic marker for detection of OPXV infection or vaccination. Consequently, most reported IgM and IgG immunoassays use the purified whole vaccinia virus as the source of viral proteins. However, culturing and purification of the virus require BSL-2 or 3 facilities and are more costly and time consuming compared to recombinant protein technology. Moreover, although the IgG-based immunoassay is useful for epidemiological surveillance in OPXV endemic areas, its utility for diagnostic purposes is limited as it can't differentiate recent Monkeypox infection or vaccinia vaccination from past smallpox vaccination. Accordingly, what is needed are affordable serologic tests for diagnosis of orthopoxvirus infection that can differentiate between infection and vaccine efficacy testing.

### BRIEF DESCRIPTION OF THE DRAWINGS

FIG. 1 shows an evaluation of recombinant A33R protein in (A) indirect anti-human IgG-CMIA and B) direct double antigen Total Ig-CMIA with 98 assumed vaccines (blood donors >50 years old) and 100 assumed non-vaccines (blood donors <40 years old).

FIG. 2 shows results evaluating A33R recombinant protein in indirect IgM and IgG assays, direct double antigen Total Ig and indirect IgG avidity assays with 5 bleeds from 3 Monkeypox infected patients and 20 assumed smallpox vaccines (US born and >50 years old)

### SUMMARY

In one embodiment, the present disclosure relates to a method of detecting a target antibody in a sample. The method comprises:
a) contacting a sample obtained from a subject with a capture antigen and a detection antigen comprising a detectable label, thereby forming a capture antigen-target antibody-detection antigen complex; and
b) detecting a target antibody in the sample based upon a signal generated by the detectable label in the capture antigen-target antibody detection antigen complex, wherein the capture antigen and detection antigen comprise a recombinant A33R, A35R, A27L, B6R, or H3L protein.

In one aspect of the above method, the capture and/or detection antigen comprise: (i) a recombinant A33R protein having at least 80% sequence identity to SEQ ID NO: 1; (ii) a recombinant A35R protein having at least 80% sequence identity to SEQ ID NO: 2; (iii) a recombinant A27L protein having at least 80% sequence identity to SEQ ID NO: 3; (iv) a recombinant B6R protein having at least 80% sequence identity to SEQ ID NO: 4; or (v) a recombinant H3L protein having at least 80% sequence identity to SEQ ID NO: 5.

In another aspect of the above method, the capture and/or detection antigen comprise: (i) a recombinant A33R protein having at least 90% sequence identity to SEQ ID NO: 1; (ii) a recombinant A35R protein having at least 90% sequence identity to SEQ ID NO: 2; (iii) a recombinant A27L protein having at least 90% sequence identity to SEQ ID NO: 3; (iv) a recombinant B6R protein having at least 90% sequence identity to SEQ ID NO: 4; or (v) a recombinant H3L protein having at least 90% sequence identity to SEQ ID NO: 5.

In another aspect of the above method, the capture and/or detection antigen comprise: (i) a recombinant A33R protein having at least 95% sequence identity to SEQ ID NO: 1; (ii) a recombinant A35R protein having at least 95% sequence identity to SEQ ID NO: 2; (iii) a recombinant A27L protein having at least 95% sequence identity to SEQ ID NO: 3; (iv) a recombinant B6R protein having at least 95% sequence identity to SEQ ID NO: 4; or (v) a recombinant H3L protein having at least 95% sequence identity to SEQ ID NO: 5.

In another aspect of the above method, the capture and/or detection antigen comprise: (i) a recombinant A33R protein having an amino acid sequence of SEQ ID NO: 1; (ii) a recombinant A35R protein having an amino acid sequence of SEQ ID NO: 2; (iii) a recombinant A27L protein having an amino acid sequence of SEQ ID NO: 3; (iv) a recombinant B6R protein having an amino acid sequence of SEQ ID NO: 4; or (v) a recombinant H3L protein having an amino acid sequence of SEQ ID NO: 5.

In another aspect of the above method, the capture antigen is bound to a solid support and the detection antigen is detectably labeled.

In still yet another aspect of the above method, the subject has or is suspected of having an orthopoxvirus infection.

In still yet another aspect of the above method, the subject has or is suspected of having a monkeypox infection.

In still yet another aspect of the above method, the orthopoxvirus vaccination status of the subject is unknown.

In still yet another aspect of the above method, the subject has received a vaccination against one or more orthopoxviruses.

In still yet another aspect of the above method, the subject is a human.

In still yet another aspect of the above method, the sample is a whole blood sample, a serum sample, or a plasma sample.

In yet another embodiment, the present disclosure relates to a method of detecting a target antibody in a sample. The method comprises:
a) contacting a sample obtained from a subject with a capture antigen, wherein the capture antigen comprises a recombinant A33R, A35R, A27L, B6R, or H3L protein;
b) contacting the sample with a detection moiety comprising a detectable label, thereby forming a capture antigen-target antibody-detection moiety complex; and
c) detecting a target antibody in the sample based upon a signal generated by the detectable label in the capture antigen-target antibody-detection moiety complex.

In one aspect of the above method, the capture antigen comprises: (i) a recombinant A33R protein having at least 80% sequence identity to SEQ ID NO: 1; (ii) a recombinant A35R protein having at least 80% sequence identity to SEQ ID NO: 2; (iii) a recombinant A27L protein having at least 80% sequence identity to SEQ ID NO: 3; (iv) a recombinant B6R protein having at least 80% sequence identity to SEQ ID NO: 4; or (v) a recombinant H3L protein having at least 80% sequence identity to SEQ ID NO: 5.

In another aspect of the above method, the capture antigen comprises: (i) a recombinant A33R protein having at least 90% sequence identity to SEQ ID NO: 1; (ii) a recombinant A35R protein having at least 90% sequence identity to SEQ ID NO: 2; (iii) a recombinant A27L protein having at least 90% sequence identity to SEQ ID NO: 3; (iv) a recombinant B6R protein having at least 90% sequence identity to SEQ ID NO: 4; or (v) a recombinant H3L protein having at least 90% sequence identity to SEQ ID NO: 5.

In still yet another aspect of the above method, the capture antigen comprises: (i) a recombinant A33R protein having at least 95% sequence identity to SEQ ID NO: 1; (ii) a recombinant A35R protein having at least 95% sequence identity to SEQ ID NO: 2; (iii) a recombinant A27L protein having at least 95% sequence identity to SEQ ID NO: 3; (iv) a recombinant B6R protein having at least 95% sequence identity to SEQ ID NO: 4; or (v) a recombinant H3L protein having at least 95% sequence identity to SEQ ID NO: 5.

In still yet another aspect of the above method, the capture antigen comprises: (i) a recombinant A33R protein having an amino acid sequence of SEQ ID NO: 1; (ii) a recombinant A35R protein having an amino acid sequence of SEQ ID NO: 2; (iii) a recombinant A27L protein having an amino acid sequence of SEQ ID NO: 3; (iv) a recombinant B6R protein having an amino acid sequence of SEQ ID NO: 4; or (v) a recombinant H3L protein having an amino acid sequence of SEQ ID NO: 5.

In yet still another aspect of the above method, the capture antigen is bound to a solid support.

In yet still another aspect of the above method, the detection moiety comprises an anti-human IgG antibody or an anti-human IgM antibody.

In still yet another aspect of the above method, the subject has or is suspected of having an orthopoxvirus infection.

In still yet another aspect of the above method, the subject has or is suspected of having a monkeypox infection.

In still yet another aspect of the above method, the orthopoxvirus vaccination status of the subject is unknown.

In still yet another aspect of the above method, the subject has received a vaccination against one or more orthopoxviruses.

In still yet another aspect of the above method, the subject is a human.

In still yet another aspect of the above method, the sample is a whole blood sample, a serum sample, or a plasma sample.

In yet another embodiment, the present disclosure relates to a method of determining the avidity of antibodies in a sample. The method comprises:
a) performing a first assay to determine the total amount of anti-A33R, anti-A35R, anti-A27L, anti-B6R, or anti-H3L antibodies in a sample obtained from a subject; and
performing a second assay to determine the amount of low avidity anti-A33R, anti-A35R, anti-A27L, anti-B6R, or anti-H3L antibodies in a sample obtained from the subject.

In one aspect of the above method, the

first assay comprises:
a) contacting a sample obtained from a subject with a solution containing a solid phase antigen such that solid phase antigen-high avidity antibody complexes and solid phase antigen-low avidity antibody complexes are formed in the sample, wherein the solid phase antigen comprises a recombinant A33R, A35R, A27L, B6R or H3L protein attached to a solid phase;
b) adding a detection moiety comprising a detectable label to the sample; and
c) measuring a signal generated by the detectable label in the sample, wherein the signal is indicative of the total amount of anti-A33R, anti-A35R, anti-A27L, anti-B6R, or anti-H3L antibodies present in the sample.

In another aspect of the above method, the second assay comprises:
a) contacting the sample with a solution containing a solid phase antigen and a liquid phase antigen such that solid phase antigen-low avidity antibody complexes and liquid phase antigen-high avidity antibody complexes are formed in the sample, wherein the solid phase antigen comprises a recombinant A33R, A35R, A27L, B6R or H3L protein attached to a solid phase and the liquid phase antigen comprises a recombinant A33R, A35R, A27L, B6R or H3L protein in solution;
b) washing the sample, thereby removing liquid phase antigen-high avidity antibody complexes from the sample;
c) adding a detection moiety comprising a detectable label to the sample; and
d) measuring a signal generated by the detectable label in the sample, wherein the signal is indicative of the amount of low avidity anti-A33R, anti-A35R, anti-A27L, anti-B6R, or anti-H3L antibodies present in the sample.

In yet another aspect, the above method further comprise determining a ratio of the signal generated by the detectable label in the second assay to the signal generated by the detectable label in the first assay.

In still yet another aspect of the above method, the method further comprises determining an avidity index for the subject, wherein the avidity index is determined by subtracting the ratio from 1, then multiplying the ratio by 100, wherein the avidity index is indicative of the amount of high-avidity anti-A33R anti-A35R, anti-A27L, anti-B6R, or anti-H3L antibodies in a sample obtained from the subject.

In yet further aspects of the above method, an avidity index of less than 45% indicates that the subject is recently infected or vaccinated with an orthopoxvirus, and wherein an avidity index of greater than 45% indicates that the subject has previously received a vaccination against one or more orthopoxviruses.

In still further aspects of the above method, the sample is a whole blood sample, a serum sample, or a plasma sample.

In still further aspects of the above method, the subject is a human.

In yet another embodiment, the present disclosure relates to a method of differentiating between an orthopoxvirus infection and previous orthopoxvirus vaccination in a subject. The method comprises:
a) performing a first assay to determine the total amount of anti-A33R, anti-A35R, anti-A27L, anti-B6R, or anti-H3L antibodies in a sample obtained from a subject;
b) performing a second assay to determine the amount of low avidity anti-A33R, anti-A35R, anti-A27L, anti-B6R, or anti-H3L antibodies in a sample obtained from the subject; and
c) differentiating between an orthopoxvirus infection and previous orthopoxvirus vaccination in the subject based upon a ratio of the amount of low avidity anti-A33R, anti-A35R, anti-A27L, anti-B6R, or anti-H3L antibodies to the amount of total anti-A33R anti-A35R, anti-A27L, anti-B6R, or anti-H3L antibodies determined in the first and second assays.

In some aspects of the above method, the first assay comprises:
a) contacting a sample obtained from a subject with a solution containing a solid phase antigen such that solid phase antigen-high avidity antibody complexes and solid phase antigen-low avidity antibody complexes are formed in the sample, wherein the solid phase antigen comprises a recombinant A33R, A35R, A27L, B6R or H3L protein attached to a solid phase;
b) adding a detection moiety comprising a detectable label to the sample, and
c) measuring a signal generated by the detectable label in the sample, wherein the signal is indicative of the total amount of anti-A33R anti-A35R, anti-A27L, anti-B6R, or anti-H3L antibodies present in the sample.

In the above method, the second assay comprises:
a) contacting the sample with a solution containing a solid phase antigen and a liquid phase antigen such that solid phase antigen-low avidity antibody complexes and liquid phase antigen-high avidity antibody complexes are formed in the sample, wherein the solid phase antigen comprises a recombinant A33R, A35R, A27L, B6R or H3L protein attached to a solid phase and the liquid phase antigen comprises a recombinant A33R, A35R, A27L, B6R or H3L protein in solution;
b) washing the sample, thereby removing liquid phase antigen-high avidity antibody complexes from the sample;
c) adding a detection moiety comprising a detectable label to the sample; and
d) measuring a signal generated by the detectable label in the sample, wherein the signal is indicative of the amount of low avidity anti-A33R, anti-A35R, anti-A27L, anti-B6R, or anti-H3L antibodies present in the sample.

In yet another aspect of the above method, the method further comprises determining a ratio of the signal generated by the detectable label in the second assay to the signal generated by the detectable label in the first assay.

In yet another aspect of the above method, the method further comprises determining an avidity index for the subject, wherein the avidity index is determined by subtracting the ratio from 1, then multiplying the ratio by 100, wherein the avidity index is indicative of the amount of high-avidity anti-A33R, anti-A35R, anti-A27L, anti-B6R, or anti-H3L antibodies in a sample obtained from the subject.

In yet another aspect of the above method, the method further comprises determining that the subject is recently infected or vaccinated with an orthopoxvirus when the avidity index is less than 45% or determining that the subject has previously received a vaccination against one or more orthopoxviruses when the avidity index of is greater than 45%.

In yet another aspect of the above method, the sample is a whole blood sample, a serum sample, or a plasma sample.

In yet another aspect of the above method, the subject is a human.

In yet another embodiment, the present disclosure relates to a system. The system comprises:
a sample receiving component configured to receive a sample from a subject;
a capture antigen and detection antigen comprising a detectable label configured to make contact with the sample to form a capture antigen-target antibody-detection antigen complex, wherein the capture/detection antigen comprises a recombinant A33R, A35R, A27L, B6R or H3L protein;
a detection component configured to measure a signal generated by the detectable label in the capture antigen-target antibody-detection antigen complex; and
an output component that indicates an amount of target antibody in the sample based on the signal.

In one aspect of the above system, the capture and/or detection antigen comprise: (i) a recombinant A33R protein having at least 80% sequence identity to SEQ ID NO: 1; (ii) a recombinant A35R protein having at least 80% sequence identity to SEQ ID NO: 2; (iii) a recombinant A27L protein having at least 80% sequence identity to SEQ ID NO: 3; (iv) a recombinant B6R protein having at least 80% sequence identity to SEQ ID NO: 4; or (v) a recombinant H3L protein having at least 80% sequence identity to SEQ ID NO: 5.

In yet another aspect of the above system, capture and/or detection antigen comprise: (i) a recombinant A33R protein having at least 90% sequence identity to SEQ ID NO: 1; (ii) a recombinant A35R protein having at least 90% sequence identity to SEQ ID NO: 2; (iii) a recombinant A27L protein having at least 90% sequence identity to SEQ ID NO: 3; (iv) a recombinant B6R protein having at least 90% sequence identity to SEQ ID NO: 4; or (v) a recombinant H3L protein having at least 90% sequence identity to SEQ ID NO: 5.

In yet another aspect of the above system, the capture and/or detection antigen comprise: (i) a recombinant A33R protein having at least 95% sequence identity to SEQ ID NO: 1; (ii) a recombinant A35R protein having at least 95% sequence identity to SEQ ID NO: 2; (iii) a recombinant A27L protein having at least 95% sequence identity to SEQ ID NO: 3; (iv) a recombinant B6R protein having at least 95% sequence identity to SEQ ID NO: 4; or (v) a recombinant H3L protein having at least 95% sequence identity to SEQ ID NO: 5.

In yet another aspect of the above system, the capture and/or detection antigen comprise: (i) a recombinant A33R protein having an amino acid sequence of SEQ ID NO: 1; (ii) a recombinant A35R protein having an amino acid sequence of SEQ ID NO: 2; (iii) a recombinant A27L protein having an amino acid sequence of SEQ ID NO: 3; (iv) a recombinant B6R protein having an amino acid sequence of SEQ ID NO: 4; or (v) a recombinant H3L protein having an amino acid sequence of SEQ ID NO: 5.

In yet another embodiment, the present disclosure relates to a system of detecting a target antibody in a sample. The system comprises:
a) contacting a sample obtained from a subject with a capture antigen, wherein the capture antigen comprises a recombinant A33R, A35R, A27L, B6R or H3L protein;
b) contacting the sample with a detection moiety comprising a detectable label, thereby forming a capture antigen-target antibody-detection moiety complex; and
detecting a target antibody in the sample based upon a signal generated by the detectable label in the capture antigen-target antibody-detection moiety complex.

In one aspect of the above system, the capture antigen comprises: (i) a recombinant A33R protein having at least 80% sequence identity to SEQ ID NO: 1; (ii) a recombinant A35R protein having at least 80% sequence identity to SEQ ID NO: 2; (iii) a recombinant A27L protein having at least 80% sequence identity to SEQ ID NO: 3; (iv) a recombinant B6R protein having at least 90% sequence identity to SEQ ID NO: 4; or (v) a recombinant H3L protein having at least 80% sequence identity to SEQ ID NO: 5.

In another aspect of the above system, the capture antigen comprises: (i) a recombinant A33R protein having at least 90% sequence identity to SEQ ID NO: 1; (ii) a recombinant A35R protein having at least 90% sequence identity to SEQ ID NO: 2; (iii) a recombinant A27L protein having at least 90% sequence identity to SEQ ID NO: 3; (iv) a recombinant B6R protein having at least 90% sequence identity to SEQ ID NO: 4; or (v) a recombinant H3L protein having at least 90% sequence identity to SEQ ID NO: 5.

In still yet another aspect of the above system, the capture antigen comprises: (i) a recombinant A33R protein having at least 95% sequence identity to SEQ ID NO: 1; (ii) a recombinant A35R protein having at least 95% sequence identity to SEQ ID NO: 2; (iii) a recombinant A27L protein having at least 95% sequence identity to SEQ ID NO: 3; (iv) a recombinant B6R protein having at least 95% sequence identity to SEQ ID NO: 4; or (v) a recombinant H3L protein having at least 95% sequence identity to SEQ ID NO: 5.

In yet another aspect of the above system, the capture antigen comprises: (i) a recombinant A33R protein having an amino acid sequence of SEQ ID NO: 1; (ii) a recombinant A35R protein having an amino acid sequence of SEQ ID NO: 2; (iii) a recombinant A27L protein having an amino acid sequence of SEQ ID NO: 3; (iv) a recombinant B6R protein having an amino acid sequence of SEQ ID NO: 4; or (v) a recombinant H3L protein having an amino acid sequence of SEQ ID NO: 5.

In yet another aspect of the above system, the detection moiety comprises an anti-human IgG antibody or an anti-human IgM antibody.

In yet another aspect of the above system, the subject has or is suspected of having an orthopoxvirus infection.

In yet another aspect of the above system, the subject has or is suspected of having a monkeypox infection.

In yet another aspect of the above system, the orthopoxvirus vaccination status of the subject is unknown.

In yet another aspect of the above system, the subject has received a vaccination against one or more orthopoxviruses.

In yet another aspect of the above system, the subject is a human.

In yet another aspect of the above system, the sample is a whole blood sample, a serum sample, or a plasma sample.

In yet another aspect of the above system, the capture antigen is bound to a solid support.

In yet another embodiment, the present disclosure relates to a system comprising:
a sample receiving component configured to receive a sample from a subject;
a sample analysis component configured to determine a total amount of anti-A33R antibodies in the sample and an amount of low avidity anti-A33R, anti-A35R, anti-A27L, anti-B6R, or anti-H3L antibodies in the sample to arrive at an index representing a relationship between the total amount of anti-A33R, anti-A35R, anti-A27L, anti-B6R, or anti-H3L antibodies and the amount of low avidity anti-A33R, anti-A35R, anti-A27L, anti-B6R, or anti-H3L antibodies; and
an output component that indicates 1) the total amount of anti-A33R, anti-A35R, anti-A27L, anti-B6R, or anti-H3L antibodies and the amount of low avidity anti-A33R, anti-A35R, anti-A27L, anti-B6R, or anti-H3L antibodies, 2) the index, 3) whether the index is above or below a preset threshold, and/or 4) whether the subject is likely infected with an orthopoxvirus or has received a vaccination against one or more orthopoxviruses.

In yet another aspect of the above system, the index is determined by subtracting the ratio from 1, then multiplying the ratio by 100, wherein the avidity index is indicative of the amount of high-avidity anti-A33R, anti-A35R, anti-A27L, anti-B6R, or anti-H3L antibodies in a sample obtained from the subject.

In yet another aspect of the above system, where an index of less than 45% indicates that the subject is recently infected or vaccinated with an orthopoxvirus, and wherein an index of greater than 45% indicates that the subject has previously received a vaccination against one or more orthopoxviruses.

In yet another aspect of the above system where the sample is a whole blood sample, a serum sample, or a plasma sample.

In yet another aspect of the above system, where the subject is a human.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Various embodiments of the methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

As used herein, the term “antibody” refers to an immunoglobulin molecule or immunologically active portion thereof, namely, an antigen-binding portion. Examples of immunologically active portions of immunoglobulin molecules include F (ab) and F(ab′)₂ fragments which can be generated, e.g., by treating an antibody with an enzyme such as pepsin. Examples of antibodies that can be used in the present disclosure include, but are not limited to, polyclonal antibodies, monoclonal antibodies, chimeric antibodies, human antibodies, humanized antibodies, recombinant antibodies, single-chain Fvs (“scFv”), affinity maturated antibodies, single chain antibodies, single domain antibodies, F (ab) fragments, F(ab′) fragments, disulfide-linked Fvs (“sdFv”), and antiidiotypic (“anti-Id”) antibodies, among others, and functionally active epitope-binding fragments of any of the above. The antibody may be of classes IgG, IgM, IgA, IgD or IgE, or fragments or derivatives thereof. The antibody may be derivatized by the attachment of one or more chemical, peptide, or polypeptide moieties known in the art. The antibody may be conjugated with a chemical moiety.

“Antibody fragment” as used herein refers to a portion of an intact antibody comprising the antigen-binding site or variable region. The portion does not include the constant heavy chain domains (i.e., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

The “area under curve” or “AUC” refers to area under a ROC curve. AUC under a ROC curve is a measure of accuracy. An AUC of 1 represents a perfect test, whereas an AUC of 0.5 represents an insignificant test. A preferred AUC may be at least approximately 0.700, at least approximately 0.750, at least approximately 0.800, at least approximately 0.850, at least approximately 0.900, at least approximately 0.910, at least approximately 0.920, at least approximately 0.930, at least approximately 0.940, at least approximately 0.950, at least approximately 0.960, at least approximately 0.970, at least approximately 0.980, at least approximately 0.990, or at least approximately 0.995.

A “receiver operating characteristic” curve or “ROC” curve refers to a graphical plot that illustrates the performance of a binary classifier system as its discrimination threshold is varied. For example, an ROC curve can be a plot of the true positive rate against the false positive rate for the different possible cutoff points of a diagnostic test. It is created by plotting the fraction of true positives out of the positives (TPR=true positive rate) vs. The fraction of false positives out of the negatives (FPR=false positive rate), at various threshold settings. TPR is also known as sensitivity, and FPR is one minus the specificity or true negative rate. The ROC curve demonstrates the tradeoff between sensitivity and specificity (any increase in sensitivity will be accompanied by a decrease in specificity); the closer the curve follows the left-hand border and then the top border of the ROC space, the more accurate the test; the closer the curve comes to the 45-degree diagonal of the ROC space, the less accurate the test; the slope of the tangent line at a cutoff point gives the likelihood ratio (LR) for that value of the test; and the area under the curve is a measure of test accuracy.

“Bead” and “particle” are used herein interchangeably and refer to a substantially spherical solid support. One example of a bead or particle is a microparticle. Microparticles that can be used herein can be any type known in the art. For example, the bead or particle can be a magnetic bead or magnetic particle. Magnetic beads/particles may be ferromagnetic, ferrimagnetic, paramagnetic, superparamagnetic or ferrofluidic. Exemplary ferromagnetic materials include Fe, Co, Ni, Gd, Dy, CrO₂, MnAs, MnBi, EuO, and NiO/Fe. Examples of ferrimagnetic materials include NiFc₂O₄, CoFc₂O₄, Fe₃O₄ (or FeO·Fe₂O₃). Beads can have a solid core portion that is magnetic and is surrounded by one or more non-magnetic layers. Alternately, the magnetic portion can be a layer around a non-magnetic core. The microparticles can be of any size that would work in the methods described herein, e.g., from about 0.75 to about 5 nm, or from about 1 to about 5 nm, or from about 1 to about 3 nm.

“Binding protein” is used herein to refer to a monomeric or multimeric protein that binds to and forms a complex with a binding partner, such as, for example, a polypeptide, an antigen, a chemical compound or other molecule, or a substrate of any kind. A binding protein specifically binds a binding partner. Binding proteins include antibodies, as well as antigen-binding fragments thereof and other various forms and derivatives thereof as are known in the art and described herein below, and other molecules comprising one or more antigen-binding domains that bind to an antigen molecule or a particular site (epitope) on the antigen molecule. Accordingly, a binding protein includes, but is not limited to, an antibody a tetrameric immunoglobulin, an IgG molecule, an IgG1 molecule, a monoclonal antibody, a chimeric antibody, a CDR-grafted antibody, a humanized antibody, an affinity matured antibody, and fragments of any such antibodies that retain the ability to bind to an antigen.

“Bispecific antibody” is used herein to refer to a full-length antibody that is generated by quadroma technology (also referred to as hybrid-hybridoma technology; see Milstein et al., Nature, 305 (5934): 537-540 (1983)), by chemical conjugation of two different monoclonal antibodies (see, Staerz et al., Nature, 314 (6012): 628-631 (1985)), or by knob-into-hole or similar approaches, which introduce mutations in the Fc region (see Holliger et al., Proc. Natl. Acad. Sci. USA, 90 (14): 6444-6448 (1993)), resulting in multiple different immunoglobulin species of which only one is the functional bispecific antibody. A bispecific antibody binds one antigen (or epitope) on one of its two binding arms (one pair of HC/LC) and binds a different antigen (or epitope) on its second arm (a different pair of HC/LC). By this definition, a bispecific antibody has two distinct antigen-binding arms (in both specificity and CDR sequences), and is monovalent for each antigen to which it binds to.

“CDR” is used herein to refer to the “complementarity determining region” within an antibody variable sequence. There are three CDRs in each of the variable regions of the heavy chain and the light chain. Proceeding from the N-terminus of a heavy or light chain, these regions are denoted “CDR1”, “CDR2”, and “CDR3”, for each of the variable regions. The term “CDR set” as used herein refers to a group of three CDRs that occur in a single variable region that binds the antigen. An antigen-binding site, therefore, may include six CDRs, comprising the CDR set from each of a heavy and a light chain variable region. A polypeptide comprising a single CDR, (e.g., a CDR1, CDR2, or CDR3) may be referred to as a “molecular recognition unit.” Crystallographic analyses of antigen-antibody complexes have demonstrated that the amino acid residues of CDRs form extensive contact with bound antigen, wherein the most extensive antigen contact is with the heavy chain CDR3. Thus, the molecular recognition units may be primarily responsible for the specificity of an antigen-binding site. In general, the CDR residues are directly and most substantially involved in influencing antigen binding.

The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as “Kabat CDRs”. Chothia and coworkers (Chothia and Lesk, J. Mol. Biol., 196:901-917 (1987); and Chothia et al., Nature, 342:877-883 (1989)) found that certain sub-portions within Kabat CDRs adopt nearly identical peptide backbone conformations, despite having great diversity at the level of amino acid sequence. These sub-portions were designated as “L1”, “L2”, and “L3”, or “H1”, “H2”, and “H3”, where the “L” and the “H” designate the light chain and the heavy chain regions, respectively. These regions may be referred to as “Chothia CDRs”, which have boundaries that overlap with Kabat CDRs. Other boundaries defining CDRs overlapping with the Kabat CDRs have been described by Padlan, FASEB J., 9:133-139 (1995), and MacCallum, J. Mol. Biol., 262 (5): 732-745 (1996). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat- or Chothia-defined CDRs.

“Identical” or “identity” as used herein in the context of two or more polypeptide or polynucleotide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation.

“Substantially identical,” as used herein may mean that a first and second sequence are at least from about 50% to about 99% identical over a region of from about 8 to about 100 or more residues (including any range within from about 8 to about 100 residues).

As used herein, the term “sample” is used in the broadest sense and generally refers to a biological material being tested for and/or suspected of containing an analyte of interest, such as a target antibody described herein. The sample may be derived from any biological source, such as, a physiological fluid, including, but not limited to, whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid, semen and so forth. In some embodiments, the sample is a whole blood sample. In some embodiments, the sample is a plasma sample. In yet other embodiments, the sample is a serum sample. The test sample may be used directly as obtained from the biological source or following a pretreatment to modify the character of the sample. For example, such pretreatment may include preparing plasma from blood, diluting viscous fluids and so forth. Methods of pretreatment may also involve filtration, precipitation, dilution, distillation, mixing, concentration, inactivation of interfering components, the addition of reagents, lysing, etc. Moreover, it may also be beneficial to modify a solid test sample to form a liquid medium or to release the analyte.

“Reference level” or “reference value” as used herein refers to an assay or cutoff value that is used to assess diagnostic (“diagnostic” cutoff), prognostic, or therapeutic efficacy and that has been linked or is associated herein with various clinical parameters (e.g., presence of disease such as, for example, to rule a subject as having a disease (“rule in”) or rule a subject as not having a disease (“rule out”)), stage of disease, severity of disease, progression, non-progression, or improvement of disease, etc.) This disclosure provides exemplary reference levels. However, it is well-known that reference levels may vary depending on the nature of the immunoassay (e.g., such as, in an immunoassay, the antibodies employed, reaction conditions, sample purity, etc.) and that assays can be compared and standardized. It further is well within the ordinary skill of one in the art to adapt the disclosure herein for other assays to obtain assay-specific reference levels for those other immunoassays based on the description provided by this disclosure. Whereas the precise value of the reference level may vary between assays, the findings as described herein should be generally applicable and capable of being extrapolated to other assays.

A “reagent” refers broadly to any agent used in a reaction, other than the analyte (e.g., nucleic acid or polypeptide being analyzed). Illustrative reagents for a nucleic acid amplification reaction include, but are not limited to, buffer, metal ions, polymerase, reverse transcriptase, primers, template nucleic acid, nucleotides, labels, dyes, nucleases, and the like. Reagents for enzyme reactions include, for example, substrates, cofactors, buffer, metal ions, inhibitors, and activators. Reagents for immunoassay include, for example, antibodies specific for a target marker, detection (e.g., labeled) antibodies, controls, buffers, and the like.

The term “label,” as used herein, refers to any atom or molecule that can be used to provide a detectable and/or quantifiable signal. In some cases, the label can be attached, directly or indirectly, to a nucleic acid or protein. Suitable labels that can be attached to probes include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

The term “dye,” as used herein, generally refers to any organic or inorganic molecule that absorbs electromagnetic radiation at a wavelength greater than or equal 340 nm.

The term “fluorescent dye,” as used herein, generally refers to any dye that emits electromagnetic radiation of longer wavelength by a fluorescent mechanism upon irradiation by a source of electromagnetic radiation, such as a lamp, a photodiode, or a laser.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

An “absolute amount” as used herein refers to the absolute value of a change or difference between at least two assay results taken or sampled at different time points and, which similar to a reference level, has been linked or is associated herein with various clinical parameters (e.g., presence of disease, stage of disease, severity of disease, progression, non-progression, or improvement of disease, etc.). “Absolute value” as used herein refers to the magnitude of a real number (such as, for example, the difference between two compared levels (such as levels taken at a first time point and levels taken at a second time point)) without regard to its sign, i.e., regardless of whether it is positive or negative.

This disclosure provides exemplary reference levels and absolute amounts (e.g., calculated by comparing reference levels at different time points). However, it is well-known that reference levels and absolute amounts may vary depending on the nature of the immunoassay (e.g., antibodies employed, reaction conditions, sample purity, etc.) and that assays can be compared and standardized. It further is well within the ordinary skill of one in the art to adapt the disclosure herein for other immunoassays to obtain immunoassay-specific reference levels and absolute amounts for those other immunoassays based on the description provided by this disclosure. Whereas the precise value of the reference level and absolute amount may vary between assays, the findings as described herein should be generally applicable and capable of being extrapolated to other assays.

“Component,” “components,” or “at least one component,” refer generally to a capture antibody, a detection or conjugate a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample, such as a patient urine, whole blood, serum or plasma sample, in accordance with the methods described herein and other methods known in the art. Some components can be in solution or lyophilized for reconstitution for use in an assay.

“Controls” as used herein generally refers to a reagent whose purpose is to evaluate the performance of a measurement system in order to assure that it continues to produce results within permissible boundaries (e.g., boundaries ranging from measures appropriate for a research use assay on one end to analytic boundaries established by quality specifications for a commercial assay on the other end). To accomplish this, a control should be indicative of patient results and optionally should somehow assess the impact of error on the measurement (e.g., error due to reagent stability, calibrator variability, instrument variability, and the like).

“Correlated to” as used herein refers to compared to.

“Derivative” of an antibody as used herein may refer to an antibody having one or more modifications to its amino acid sequence when compared to a genuine or parent antibody and exhibit a modified domain structure. The derivative may still be able to adopt the typical domain configuration found in native antibodies, as well as an amino acid sequence, which is able to bind to targets (antigens) with specificity. Typical examples of antibody derivatives are antibodies coupled to other polypeptides, rearranged antibody domains, or fragments of antibodies. The derivative may also comprise at least one further compound, e.g., a protein domain, said protein domain being linked by covalent or non-covalent bonds. The linkage can be based on genetic fusion according to the methods known in the art. The additional domain present in the fusion protein comprising the antibody may be linked by a flexible linker, advantageously a peptide linker, wherein said peptide linker comprises plural, hydrophilic, peptide-bonded amino acids of a length sufficient to span the distance between the C-terminal end of the further protein domain and the N-terminal end of the antibody or vice versa. The antibody may be linked to an effector molecule having a conformation suitable for biological activity or selective binding to a solid support, a biologically active substance (e.g., a cytokine or growth hormone), a chemical agent, a peptide, a protein, or a drug, for example.

“Sensitivity” refers to the proportion of subjects for whom the outcome is positive that are correctly identified as positive (e.g., correctly identifying those subjects with a disease or medical condition for which they are being tested). For example, this might include correctly identifying subjects as having an orthopoxvirus infection (e.g. monkeypox infection) from those who do not have an infection. As another example, this may include correctly identifying subjects as having received a vaccinia vaccine from those who have not.

“Specificity” of an assay as used herein refers to the proportion of subjects for whom the outcome is negative that are correctly identified as negative (e.g., correctly identifying those subjects who do not have a disease or medical condition for which they are being tested). For example, this might include correctly identifying subjects not having an orthopoxvirus infection (e.g. not having monkeypox infection) from those who do have an orthopoxvirus infection. As another example, this may include correctly identifying subjects as not having received a vaccinia vaccine from those who have received the vaccine.

“Subject” and “patient” as used herein interchangeably refers to any vertebrate, including, but not limited to, a mammal and a human. In some embodiments, the subject may be a human or a non-human. The subject or patient may be undergoing forms of treatment. “Mammal” as used herein refers to any member of the class Mammalia, including, without limitation, humans and nonhuman primates such as chimpanzees and other apes and monkey species; farm animals such as cattle, sheep, pigs, goats, llamas, camels, and horses; domestic mammals such as dogs and cats; laboratory animals including rodents such as mice, rats, rabbits, guinea pigs, and the like. The term does not denote a particular age or sex. Thus, adult and newborn subjects, as well as fetuses, whether male or female, are intended to be included within the scope of this term.

“Treat,” “treating” or “treatment” are each used interchangeably herein to describe reversing, alleviating, or inhibiting the progress of a disease and/or injury, or one or more symptoms of such disease, to which such term applies. In some aspects, a treatment may be either performed in an acute or chronic way. Depending on the condition of the subject, the term also refers to preventing a disease or injury, and includes preventing the onset of a disease or injury, or preventing the symptoms associated with a disease or injury. “preventing” also refers to preventing the recurrence of a disease or injury or of one or more symptoms associated with such disease or injury. “Treatment” and “therapeutically,” refer to the act of treating, as “treating” is defined above. In some aspects, the prevention or treatment of a disease can be done prior to affliction or injury, such as, for example, to reduce the severity of a disease or injury or symptoms associated with a disease or an injury. Such prevention or reduction can include (a) administration of one or more pharmaceutical composition and/or one or more nutritional compositions to a subject; (b) the use of one or more of physical therapy, occupational therapy, and/or counseling; or (c) any combinations of (a) and (b).

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Immunoassays for Detecting and/or Diagnosing Orthopoxvirus Infection in a Subject

In some aspects, provided herein are immunoassays. In some embodiments, provided herein are immunoassays for detecting a target antibody in a sample obtained from a subject. In some embodiments, provided herein are immunoassays for detecting antibodies elicited by infection with orthopoxvirus (e.g. monkeypox) and/or vaccination with vaccinia virus (e.g. vaccination against smallpox) in a sample obtained from a subject. In some embodiments, the immunoassays provided herein are serologic assays. The term “serologic assay” or “serologic” as used herein refers to an assay that detects antibodies or antibody fragments in a sample. Broadly speaking, the immunoassays (i.e. serologic assays) provided herein comprise contacting a sample obtained from a subject with a capture moiety that binds to a target antibody in the sample. In some embodiments, the capture moiety is a capture antigen. In some embodiments, the immunoassays provided herein comprise contacting the sample with an antigen (e.g. a capture antigen) that binds to a target antibody (Ab) in a sample.

The immunoassays may be employed in a direct or an indirect assay format. A “direct assay” or a “direct immunoassay” refers to an assay in which at least two antigens (e.g. the antigens that bind to the target antibody in the sample) are incorporated (e.g., coated) on to a solid phase. At least one of the antigens is used to capture the target antibody and is referred to as a “capture antigen” (Ag). The at least one other antigen is labeled with a detectable label and is referred to as the “detection antigen” (Ag*). Accordingly, binding of both antigens to the target antibody forms a capture antibody (Ag)-target antibody-detection antigen (Ag*) complex that produces a detectable signal. Accordingly, in some embodiments the complex resulting from binding of the antigen to the target antibody is also referred to herein as a capture antigen-target antibody complex. In contrast, an “indirect assay” or an “indirect immunoassay” utilizes a capture antigen and a separate detection moiety. The capture antigen binds to the target antibody in the sample, forming a capture antigen-target antibody complex, and the detection moiety (which is detectably labeled) binds to the capture antigen-target antibody complex, thereby forming a capture antigen-target antibody-detection moiety complex that produces a detectable signal. The sample can be contacted with the capture antigen and the detection moiety either simultaneously or sequentially, in any order.

Commonly used serologic tests for detecting antibodies elicited by orthopoxvirus infection and/or vaccination with vaccinia virus (e.g., smallpox vaccination) use purified whole vaccinia virus as the source of viral proteins. In contrast, the immunoassays described herein are predicated at least in part on the discovery of specific proteins that encompass the immunodominant response elicited by monkeypox infection. Accordingly, the immunoassays described herein can be performed without requiring use of a whole purified virus (e.g. a whole purified vaccinia virus) as the source of viral proteins to detect antibodies in a sample. Specifically, it was discovered herein that antibodies against the monkeypox viral protein A33R (anti-A33R), the monkeypox viral protein A35R (anti-A35R) the monkeypox viral protein A27L, monkeypox viral protein B6R (anti-B6R), monkeypox viral protein H3L (anti-H3L), or any combinations thereof, are the immunodominant responses elicited as a result of monkeypox infection and/or vaccinia vaccination in a subject.

In some aspects, the immunoassays provided herein comprise contacting a sample with an antigen (e.g. a capture antigen) that binds to an antibody against the monkeypox protein A33R. The A33R gene of monkeypox virus (Uniprot accession no NP_536572) encodes a protein having the following sequence:

MNQCMSANEAAITDSAVAVAAASSTHRKVASSTTQYDHKESCNGLYYQGSC YILHSDYKSFEDAKANCAAESSTLPNKSDVLTTWLIDYVEDTWGSDGNPITKTTSDYQD SDVSQEVRKYFCT (SEQ ID NO: 1). In some embodiments, the immunoassays provided herein comprise contacting the sample with antigen that binds to an antibody against SEQ ID NO: 1. In some embodiments, the antigen (e.g. the antigen that binds to an antibody against SEQ ID NO: 1) is a recombinant A33R protein. In some embodiments, the antigen is a recombinant A33R protein having at least 80% sequence identity to SEQ ID NO: 1. In some embodiments, the antigen is a recombinant A33R protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 1. In some aspects, the antigen is a recombinant A33R protein having at least 80% to at least 95% or at least 85% to at least 95% identity to SEQ ID NO:1.

In some embodiments, the immunoassay comprise contacting the sample with a recombinant A33R protein having at least 80% sequence identity to SEQ ID NO: 1. In some embodiments, the immunoassay comprises contacting the sample with a recombinant A33R protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 1. In some aspects, the immunoassay comprises contacting the sample with a recombinant A33R protein having at least 80% to at least 95% or at least 85% to at least 95% identity to SEQ ID NO:1.

In some embodiments, the immunoassays provided herein comprise contacting a sample with an antigen (e.g. a capture antigen) that binds to an antibody against the monkeypox protein A35R. The A35R gene of monkeypox virus (Uniprot accession no. EPI_ISL_13052264-Arg58-Thr181) encodes a protein having the following sequence:

RLNQCMSANKAAITDSAVAVAAASSTHRKVVSSTTQYDHKESCNGLYYQGS CYILHSDYKSFEDAKANCAAESSTLPNKSDVLTTWLIDYVEDTWGSDGNPITKTTSDYQ DSDVSQEVRKYFCT (SEQ ID NO: 2). In some embodiments, the immunoassays provided herein comprise contacting the sample with antigen that binds to an antibody against SEQ ID NO: 2. In some embodiments, the antigen (e.g. the antigen that binds to an antibody against SEQ ID NO: 2) is a recombinant A35R protein. In some embodiments, the antigen is a recombinant A35R protein having at least 80% sequence identity to SEQ ID NO: 2. In some embodiments, the antigen is a recombinant A35R protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 2. In some aspects the antigen is a recombinant A35R protein having at least 80% to at least 95% or at least 85% to at least 95% identity to SEQ ID NO:2.

In some embodiments, the immunoassay comprise contacting the sample with a recombinant A35R protein having at least 80% sequence identity to SEQ ID NO: 2. In some embodiments, the immunoassay comprises contacting the sample with a recombinant A35R protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 2. In some aspects, the immunoassay comprises contacting the sample with a recombinant A35R protein having at least 80% to at least 95% or at least 85% to at least 95% identity to SEQ ID NO:2. In some embodiments, the immunoassays provided herein comprise contacting a sample with an antigen (e.g. a capture antigen) that binds to an antibody against the vaccina protein A27L. The A27L gene of Vaccinia virus (VACV) (strain Copenhagen) virus (Uniprot accession no. P20535-Met1-Glu110) encodes a protein having the following sequence:

MDGTLFPGDDDLAIPATEFFSTKADKKPEAKREAIVKADEDDNEETLKQRLTN LEKKITNVTTKFEQIEKCCKRNDEVLFRLENHAETLRAAMISLAKKIDVQTGRRPYE (SEQ ID NO: 3). In some embodiments, the immunoassays provided herein comprise contacting the sample with antigen that binds to an antibody against SEQ ID NO: 3. In some embodiments, the antigen (e.g. the antigen that binds to an antibody against SEQ ID NO: 3) is a recombinant A27L protein. In some embodiments, the antigen is a recombinant A27L protein having at least 80% sequence identity to SEQ ID NO: 3. In some embodiments, the antigen is a recombinant A27L protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 3. In some aspects the antigen is a recombinant A27L protein having at least 80% to at least 95% or at least 85% to at least 95% identity to SEQ ID NO:3.

In some embodiments, the immunoassay comprise contacting the sample with a recombinant A27L protein having at least 80% sequence identity to SEQ ID NO: 3. In some embodiments, the immunoassay comprises contacting the sample with a recombinant A27L protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 3. In some aspects the immunoassay comprises contacting the sample with a recombinant A27L protein having at least 80% to at least 95% or at least 85% to at least 95% identity to SEQ ID NO:3.

In some embodiments, the immunoassays provided herein comprise contacting a sample with an antigen (e.g. a capture antigen) that binds to an antibody against the monkeypox protein B6R. The B6R gene of monkeypox virus (Uniprot accession no. QNP13760.1-Tyr18-His279) encodes a protein having the following sequence:

YSTCTVPTMNNAKLTSTETSFNDKQKVTFTCDSGYHSLDPNAVCETDKWKYE NPCKKMCTVSDYVSELYDKPLYEVNSTMTLSCNGETKYFRCEEKNGNTSWNDTVTCPN AECQPLQLEHGSCQPVKEKYSFGEYMTINCDVGYEVIGVSYISCTANSWNVIPSCQQKC DIPSLSNGLISGSTFSIGGVIHLSCKSGFTLTGSPSSTCIDGKWNPILPTCVRSNEEFDPVDD GPDDETDLSKLSKDVVQYEQEIESLEATYH (SEQ ID NO: 4). In some embodiments, the immunoassays provided herein comprise contacting the sample with antigen that binds to an antibody against SEQ ID NO: 4. In some embodiments, the antigen (e.g. the antigen that binds to an antibody against SEQ ID NO: 4) is a recombinant B6R protein. In some embodiments, the antigen is a recombinant B6R protein having at least 80% sequence identity to SEQ ID NO: 4. In some embodiments, the antigen is a recombinant B6R protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 4. In some aspects the antigen is a recombinant B6R protein having at least 80% to at least 95% or at least 85% to at least 95% identity to SEQ ID NO:4.

In some embodiments, the immunoassay comprise contacting the sample with a recombinant B6R protein having at least 80% sequence identity to SEQ ID NO: 4. In some embodiments, the immunoassay comprises contacting the sample with a recombinant B6R protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 4. In some aspects the immunoassay comprises contacting the sample with a recombinant B6R protein having at least 80% to at least 95% or at least 85% to at least 95% identity to SEQ ID NO:4.

In some embodiments, the immunoassays provided herein comprise contacting a sample with an antigen (e.g. a capture antigen) that binds to an antibody against the monkeypox protein H3L. The H3L gene of monkeypox virus (Uniprot accession no. QNP13688.1-Met1-Pro278) encodes a protein having the following sequence:

MAAVKTPVIVVPVIDRPPSETFPNVHEHINDQKFDDVKDNEVMQEKRDVVIV NDDPDHYKDYVFIQWTGGNIRDDDKYTHFFSGFCNTMCTEETKRNIARHLALWDSKFF TELENKNVEYVVIIENDNVIEDITFLRPVLKAIHDKKIDILQMREIITGNKVKTELVIDKDH AIFTYTGGYDVSLSAYIIRVTTALNIVDEIIKSGGLSSGFYFEIARIENEMKINRQIMDNSA KYVEHDPRLVAEHRFETMKPNFWSRIGTVAAKRYPGVMYTFTTP (SEQ ID NO: 5). In some embodiments, the immunoassays provided herein comprise contacting the sample with antigen that binds to an antibody against SEQ ID NO: 5. In some embodiments, the antigen (e.g. the antigen that binds to an antibody against SEQ ID NO: 5) is a recombinant H3L protein. In some embodiments, the antigen is a recombinant H3L protein having at least 80% sequence identity to SEQ ID NO: 5. In some embodiments, the antigen is a recombinant H3L protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 5. In some aspects the antigen is a recombinant H3L protein having at least 80% to at least 95% or at least 85% to at least 95% identity to SEQ ID NO:5.

In some embodiments, the immunoassay comprises contacting the sample with a recombinant H3L protein having at least 80% sequence identity to SEQ ID NO: 5. In some embodiments, the immunoassay comprises contacting the sample with a recombinant H3L protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 5. In some aspects the immunoassay comprises contacting the sample with a recombinant H3L protein having at least 80% to at least 95% or at least 85% to at least 95% identity to SEQ ID NO:5.

In some embodiments, provided herein are direct immunoassays. In some embodiments, provided herein is a direct immunoassay wherein the capture antigen binds to an antibody against A33R, A35R, A27L, B6R, H3L or any combinations thereof). In some embodiments, provided herein is a direct immunoassay comprising contacting the sample with a capture antigen that binds to an antibody against SEQ ID NO: 1 SEQ ID NO: 2, SEQ ID NO: 3, SEQ ID NO: 4, SEQ ID NO: 5 or any combinations thereof. In some embodiments, the capture antigen is a recombinant A33R protein. In some embodiments, the capture antigen is a recombinant A33R protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 1. In some embodiments the capture antigen is a recombinant A33R protein having at least 80% to at least 95% or at least 85% to at least 95% identity to SEQ ID NO:1. In some aspects, the capture antigen is a recombinant A35R protein. In some embodiments, the capture antigen is a recombinant A35R protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 2. In some embodiments the capture antigen is a recombinant A35R protein having at least 80% to at least 95% or at least 85% to at least 95% identity to SEQ ID NO:2. In some aspects, the capture antigen is a recombinant A27L protein. In some embodiments, the capture antigen is a recombinant A27L protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 3. In some embodiments the capture antigen is a recombinant A27L protein having at least 80% to at least 95% or at least 85% to at least 95% identity to SEQ ID NO:3.

In some aspects, the capture antigen is a recombinant B6R protein. In some embodiments, the capture antigen is a recombinant B6R protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 4. In some embodiments the capture antigen is a recombinant B6R protein having at least 80% to at least 95% or at least 85% to at least 95% identity to SEQ ID NO:4.

In some aspects, the capture antigen is a recombinant H3L protein. In some embodiments, the capture antigen is a recombinant H3L protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 5. In some embodiments the capture antigen is a recombinant H3L protein having at least 80% to at least 95% or at least 85% to at least 95% identity to SEQ ID NO:5.

The detection antigen used in the direct immunoassay can any of recombinant A33R, A35R, A27L, B6R, H3L or any combinations thereof that is labeled with a detectable label such that when the detection antigen binds to the antibody of interest (i.e. a target antibody) produces a detectable signal. In some embodiments, provided herein is a direct immunoassay comprising contacting the sample with a detection antigen that binds to an antibody against SEQ ID NO: 1 SEQ ID NO: 2, SEQ ID NO: 3, SEQ ID NO: 4, SEQ ID NO: 5 or any combinations thereof, wherein the detection antigen is detectably labeled. In some embodiments, the detection antigen is a recombinant A33R protein. In some embodiments, the detection antigen is a recombinant A33R protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 1. In some embodiments the detection antigen is a recombinant A33R protein having at least 80% to at least 95% or at least 85% to at least 95% identity to SEQ ID NO:1. In some aspects, the detection antigen is a recombinant A35R protein. In some embodiments, the detection antigen is a recombinant A35R protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 2. In some embodiments the detection antigen is a recombinant A35R protein having at least 80% to at least 95% or at least 85% to at least 95% identity to SEQ ID NO:2. In some aspects, the detection antigen is a recombinant A27L protein. In some embodiments, the detection antigen is a recombinant A27L protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 3. In some embodiments the detection antigen is a recombinant A27L protein having at least 80% to at least 95% or at least 85% to at least 95% identity to SEQ ID NO:3.

In some aspects, the detection antigen is a recombinant B6R protein. In some embodiments, the detection antigen is a recombinant B6R protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 4. In some embodiments the detection antigen is a recombinant B6R protein having at least 80% to at least 95% or at least 85% to at least 95% identity to SEQ ID NO:4.

In some aspects, the detection antigen is a recombinant H3L protein. In some embodiments, the detection antigen is a recombinant H3L protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 5. In some embodiments the detection antigen is a recombinant H3L protein having at least 80% to at least 95% or at least 85% to at least 95% identity to SEQ ID NO:5.

In some aspects, the capture and detection antigens used in the direct assay are the same (e.g., recombinant A33R protein is used both as the capture antigen and detection antigen, recombinant A35R protein is used as both the capture antigen and detection antigen, recombinant A27L protein is used as both the capture antigen and detection antigen, recombinant B6R protein is used as both the capture antigen and detection antigen, or recombinant H3L protein is used both as the capture antigen and detection antigen). In some embodiments, provided herein are indirect immunoassays. In some embodiments, provided herein is an indirect immunoassay involving the use of separate capture and detection moieties, wherein the capture moiety (e.g. the capture antigen) binds to a target of interest forming a capture antigen-target antibody complex, and the detection antigen binds to the capture antigen-target antibody complex. In some embodiments, the detection moiety comprises a detectable label, such that binding of the detection moiety to the capture antigen-target antibody complex produces a detectable signal. In some embodiments, the detection moiety (e.g. the detection antibody) binds to the target antibody in the capture antigen-target antibody complex.

In some embodiments, provided herein are indirect immunoassays comprising contacting the sample with a capture antigen and a detection moiety. In some embodiments, the capture antigen is a recombinant A33R protein. In some embodiments, the capture antigen is a recombinant A33R protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 1. In some embodiments, the detection moiety is an antibody of fragment thereof. In some embodiments, the detection moiety is an anti-human IgG antibody or an anti-human IgM antibody.

In some embodiments, the capture antigen is a recombinant A33R protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 1 and the detection moiety is an anti-human IgG antibody.

In some embodiments, the capture antigen is a recombinant A33R protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 1 and the detection moiety is an anti-human IgM antibody.

In some embodiments, the capture antigen is a recombinant A35R protein. In some embodiments, the capture antigen is a recombinant A35R protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 2. In some embodiments, the detection moiety is an antibody of fragment thereof. In some embodiments, the detection moiety is an anti-human IgG antibody or an anti-human IgM antibody.

In some embodiments, the capture antigen is a recombinant A35R protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 2 and the detection moiety is an anti-human IgG antibody.

In some embodiments, the capture antigen is a recombinant A35R protein. In some embodiments, the capture antigen is a recombinant A35R protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 2 and the detection moiety is an anti-human IgM antibody.

In some embodiments, the capture antigen is a recombinant A27L protein. In some embodiments, the capture antigen is a recombinant A27L protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 3. In some embodiments, the detection moiety is an antibody of fragment thereof. In some embodiments, the detection moiety is an anti-human IgG antibody or an anti-human IgM antibody.

In some embodiments, the capture antigen is a recombinant A27L protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 3 and the detection moiety is an anti-human IgG antibody.

In some embodiments, the capture antigen is a recombinant A27L protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 3 and the detection moiety is an anti-human IgM antibody.

In some embodiments, the capture antigen is a recombinant B6R protein. In some embodiments, the capture antigen is a recombinant B6R protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 4. In some embodiments, the detection moiety is an antibody of fragment thereof. In some embodiments, the detection moiety is an anti-human IgG antibody or an anti-human IgM antibody.

In some embodiments, the capture antigen is a recombinant B6R protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 4 and the detection moiety is an anti-human IgG antibody.

In some embodiments, the capture antigen is a recombinant B6R protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 4 and the detection moiety is an anti-human IgM antibody.

In some embodiments, the capture antigen is a recombinant H3L protein. In some embodiments, the capture antigen is a recombinant H3L protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 5. In some embodiments, the detection moiety is an antibody of fragment thereof. In some embodiments, the detection moiety is an anti-human IgG antibody or an anti-human IgM antibody.

In some embodiments, the capture antigen is a recombinant H3L protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 5 and the detection moiety is an anti-human IgG antibody.

In some embodiments, the capture antigen is a recombinant H3L protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 5 and the detection moiety is an anti-human IgM antibody.

In some embodiments, the immunoassay measures avidity of an antibody-antigen interaction. Such an assay is referred to herein as an “avidity assay” or an “avidity immunoassay”. The “avidity” or “functional affinity” of an antibody-antigen interaction is a measurement of the strength of interactions between the antibody and the antigen. The avidity of virus specific IgG antibodies is usually low during primary viral infection and increases over time. Accordingly, avidity can be used to differentiate recent infection from past infections or vaccinations that produce an antibody response. In particular, the avidity assays described herein are based at least in part on the observation that low avidity IgG antibodies produced during acute infection with orthopoxvirus have a low affinity for a “liquid phase antigen” and a high affinity for same “solid phase antigen”. In contrast, high avidity IgG antibodies, such as those resulting from a previous vaccination against orthopoxvirus, have a preferential affinity for “liquid phase antigen” under optimal assay conditions, including optimal dilution of the patient sample. Thus, the avidity assays described herein use the differential affinity of low and high avidity antibodies to “solid phase antigen” and same “liquid phase antigen” in order to distinguish between antibodies of low and high avidity and thereby discriminate at the diagnostic level between an acute infection with orthopoxvirus (e.g. monkeypox) and a previous vaccination against one or more orthopoxviruses.

As used herein, the term “liquid phase antigen” refers to an antigen in solution, which comprises one or more epitopes that bind to an antibody also freely mobile within a solution. In contrast, a “solid phase antigen” is defined as an antigen that is attached to a solid phase, which comprises one or more epitopes that can capture an antibody in solution. A “solid phase” may be a porous or non-porous material, a latex particle, a magnetic particle, a microparticle, a bead, a membrane, and a microtiter well or a plastic tube.

In some embodiments, the immunoassay is a competitive immunoassay that measures the avidity of antibodies in a sample. For example, in some embodiments the immunoassay is a competitive immunoassay that measures the avidity of anti-A33R antibodies in a sample. In some embodiments, the immunoassay (e.g. the competitive immunoassay) uses a recombinant A33R, A35R, A27L, B6R, or H3L protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NOS: 1, 2, 3, 4, or 5, respectively, as the capture antigen to form the solid phase antigen and the liquid phase antigen. In some embodiments, the competitive immunoassay uses an anti-human IgG antibody as a detection moiety.

In some embodiments, the competitive immunoassay comprises two assays, a first assay involving contacting a sample with a solid phase antigen and a second assay involving contacting a sample with a solid phase and liquid phase antigen. In some embodiments, the results of the two assays are compared in order to generate an avidity index.

In some embodiments, the first assay comprises contacting a sample with a solution comprising a solid phase antigen. In some embodiments, the solid phase antigen comprises a recombinant A33R, A35R, A27L, B6R, or H3L protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NOS: 1, 2, 3, 4 or 5, respectively, attached to a solid phase (e.g. a microparticle). In some embodiments, the solution does not contain a liquid phase antigen (e.g. does not contain a recombinant A33R protein in solution). Accordingly, in the first assay, both low avidity and high avidity antibodies (e.g. low avidity and high avidity anti-A33R, anti-A35R, anti-A27L, anti-B6R or anti-H3L antibodies) will bind to the solid phase antigen, thereby forming solid phase antigen-low avidity antibody complexes and solid phase antigen-high avidity antibody complexes in the sample. In some embodiments, following incubation of the sample with the solution comprising a solid phase antigen a detection moiety is added, which binds to the antibodies bound to the solid phase antigen. As described herein, in some embodiments the detection moiety comprises a detectable label. The antibodies bound to the solid phase antigen are also referred to as a capture antigen-target antibody complex. The detection moiety binds to the capture antigen-target antibody complexes (e.g. the solid phase antigen-low avidity antibody complexes and the solid phase antigen-high avidity antibody complexes) in the sample, thereby producing a detectable signal. In this first assay, the amount of detectable signal is a reflection of the amount of both low avidity antibodies (e.g. low avidity anti-A33R, anti-A35R, anti-A27L, anti-B6R or anti-H3L antibodies) and high avidity antibodies (e.g. high avidity anti-A33R antibodies) in the sample, given that both low and high avidity antibodies will bind to the solid phase antigen.

In some embodiments, the second assay comprises contacting a sample with a solution comprising a liquid phase antigen (e.g. a recombinant A33R, A35R, A27L, B6R, or H3L protein in solution) and a solid phase antigen (e.g. a recombinant A33, A35R, A27L, B6R, or H3L protein attached to a solid phase). For example, in some embodiments the second assay comprises contacting the sample with a solution comprising a recombinant A33R A35R, A27L, B6R, or H3L protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NOS: 1, 2, 3, 4 or 5, respectively, attached to a solid phase (i.e. a solid phase antigen) and a solution comprising a recombinant A33R, A35R, A27L, B6R, or H3L protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NOS: 1, 2, 3, 4, or 5, respectively, in solution (i.e. a liquid phase antigen). In the second assay, low avidity antibodies (e.g. low avidity anti-A33R, anti-A35R, anti-A27L, anti-B6R or anti-H3L antibodies) preferentially bind to the solid phase antigen, thereby producing solid phase antigen-low avidity antibody complexes, whereas high avidity antibodies (e.g. high avidity anti-A33R, anti-A35R, anti-A27L, anti-B6R or anti-H3L antibodies) preferentially bind to the liquid phase antigen, thereby forming liquid phase antigen-high avidity antibody complexes. In some embodiments, the second assay comprises a wash step, which removes antibodies not bound to the solid phase antigen (e.g. removes the liquid phase antigen-high avidity antibody complexes from the sample). In some embodiments, following the wash step a detection moiety is added, which binds to the antibodies bound to the solid phase antigen (e.g. binds to the solid phase antigen-low avidity antibody complexes). As described herein, in some embodiments the detection moiety comprises a detectable label. The antibodies bound to the solid phase antigen are also referred to as a capture antigen-target antibody complex. The detection moiety binds to the capture antigen-target antibody complex (e.g. the solid phase antigen-low avidity antibody complexes), thereby producing a detectable signal. In this second assay, the amount of detectable signal is a reflection of the amount of low avidity antibodies (e.g. low avidity anti-A33R antibodies) in the sample.

In some embodiments, the results of the first assay and the second assay are compared in order to determine the avidity of the sample. The ratio of the signal obtained in the second assay (i.e. assay #2) over the signal in the first assay (i.e. assay #1) is proportional to the level low avidity antibodies present in the sample. Increased levels of low avidity antibodies in the sample will lead to a higher signal in assay #2, and therefore a higher ratio of the signal in the first assay compared to the signal in the first assay.

In some embodiments, an avidity index of the sample is calculated. The avidity index is a reflection of the proportion of high avidity antibodies in the sample. In some embodiments, the avidity index of the sample is calculated by mathematically transforming the results of the first and second assay as follows:

$$Avidity index = [ 1 - ( signal assay \#2 / signal assay \#1 ) ] \times 100.$$

The avidity index is indicative of the amount of high-avidity anti-A33R, anti-A35R, anti-A27L, anti-B6R, or anti-H3L antibodies in a sample obtained from a subject. In some aspects, as shown in the above formula, the avidity index is determined by first determining the ratio of signal generated by the detectable label in the second assay (assay #2) to the signal generated by the detectable label in a first assay (assay #1). Once the ratio is determined, then it is subtracted from 1 and then multiplied by 100.

The avidity index is proportional to the amount of high avidity antibodies present in the sample, with a lower avidity index indicating fewer high avidity antibodies present in the sample and a higher avidity index indicating greater numbers of high avidity antibodies present in the sample. Accordingly, in some embodiments a high avidity index is indicative of a previous vaccination against orthopoxvirus, which generates increasing numbers of high avidity antibodies over time. In contrast, in some embodiments a low avidity index is indicative of current or recent orthopoxvirus infection, which generates low avidity antibodies in response to the infection.

In some embodiments, the capture moiety (e.g. capture antigen) is bound to a solid support or solid phase. In some embodiments, the solid support or solid phase facilitates separation of the capture antigen-target antibody complex from the test sample. Any solid support known in the art can be used, including but not limited to, solid supports made out of polymeric materials in the forms of wells of a reaction tray, test tubes or beads (for example, polystyrene beads, magnetic beads), nitrocellulose strips, membranes, microparticles (for example, latex particles, sheep and DURACYTES® (Abbott Laboratories, Abbott Park, IL; DURACYTES® are red blood cells that have been “fixed” by pyruvic aldehyde and formaldehyde)).

The solid phase also can comprise any suitable porous material with sufficient porosity and surface affinity. Microporous structures are generally used, but materials with gel structure in the hydrated state may be used as well. Such useful solid supports include, but are not limited to, nitrocellulose and nylon. Such porous solid supports are in the form of sheets of thickness from about 0.01 to 0.5 mm, including about 0.1 mm. The pore size may vary within wide limits, and can be from about 0.025 to about 15 microns, especially from about 0.15 to about 15 microns. The surface of such supports may be activated by chemical processes which cause covalent linkage of the antigen or antibody to the support. The irreversible binding of the antigen or antibody is obtained, however, in general, by adsorption on the porous material by poorly understood hydrophobic forces.

The capture moiety (e.g. capture antigen) can be bound to the solid support or solid phase by adsorption, by covalent bonding using a chemical coupling agent or by other means known in the art, provided that such binding does not interfere with the ability of the antigen to bind to the target antibody. Alternatively, the capture antigen can be bound to microparticles that have previously coated with streptavidin or biotin (for example, using Power-Bind™-SA-MP streptavidin coated microparticles, available from Seradyn, Indianapolis, Indiana) with biotinylated moieties using means known in the art. Alternatively, the capture antigen can be bound using microparticles that have been previously coated with anti-species specific monoclonal antibodies. Moreover, if necessary, the solid support can be derivatized to allow reactivity with various functional groups on the capture antigen. Such derivatization requires the use of certain coupling agents such as, but not limited to, maleic anhydride, N-hydroxysuccinimide and 1-ethyl-3-(3-dimethylaminopropyl) carbodiimide.

After the test sample being tested for and/or suspected of containing the marker is brought into contact with the capture moiety (e.g. the capture antigen), the mixture is incubated in order to allow for the formation of a capture antigen-target antibody complex. The incubation can be carried out at a pH of from about 4.5 to about 10.0, at a temperature of from about 2° C. to about 45° C., and for a period from at least about one (1) minute to about eighteen (18) hours, including from about 1 to 20 minutes, also including from about 2-6 minutes. The immunoassay described herein can be conducted in one step (meaning the test sample, at least one capture antigen and at least one detection moiety are all added sequentially or simultaneously to a reaction vessel) or in more than one step, such as two steps, three steps, etc.

In some embodiments, after formation of the capture antigen-antibody complex, the complex is then contacted with at least one detection moiety under conditions such that the detection moiety binds to the capture antigen-target antibody complex, thereby forming a capture antigen-target antibody-detection moiety complex. In some embodiments, multiple detection moieties are used. If the capture antigen-target moiety complex is contacted with more than one detection antibody, then multiple capture antigen-target antibody-detection moiety complexes are formed.

In some embodiments, the capture moiety (e.g. the capture antigen) contains a detectable label. In some embodiments, the detectable label is bound to the capture moiety prior to, simultaneously with, or after formation of the capture antigen-target antibody complex. In some embodiments, the detection moiety contains a detectable label. The detectable label can be bound to the detection moiety prior to, simultaneously with, or after the formation of the capture antigen-target antibody-detection moiety complex.

Any detectable label known in the art can be used. For example, the detectable label can be a radioactive label, such as, ³H, ¹²⁵I, ³⁵S, ¹⁴C, ³²P, ³³P, an enzymatic label, such as horseradish peroxidase, alkaline phosphatase, glucose 6-phosphate dehydrogenase, etc., a chemiluminescent label, such as, acridinium (e.g., acridium esters, acridinium SPSP (N10-(3-sulfopropyl)-N-(3-sulfopropyl, etc.), luminol, isoluminol, thioesters, sulfonamides, phenanthridinium esters, etc. a fluorescence label, such as, fluorescein (5-fluorescein, 6-carboxyfluorescein, 3′6-carboxyfluorescein, 5 (6)-carboxyfluorescein, 6-hexachloro-fluorescein, 6-tetrachlorofluorescein, fluorescein isothiocyanate, etc.), rhodamine, phycobiliproteins, R-phycoerythrin, quantum dots (zinc sulfide-capped cadmium selenide), a thermometric label or an immuno-polymerase chain reaction label. An introduction to labels, labeling procedures and detection of labels is found in Polak and Van Noorden, Introduction to Immunocytochemistry, 2^{nd} ed., Springer Verlag, N.Y. (1997) and in Haugland, Handbook of Fluorescent Probes and Research Chemicals (1996), which is a combined handbook and catalogue published by Molecular Probes, Inc., Eugene, Oregon.

The detectable label can be bound to the capture moiety or the detection moiety either directly or through a coupling agent. An example of a coupling agent that can be used is EDAC (1-ethyl-3-(3-dimethylaminopropyl) carbodiimide, hydrochloride) that is commercially available from Sigma-Aldrich, St. Louis, MO. Other coupling agents that can be used are known in the art. Methods for binding a detectable label to an antibody are known in the art. Additionally, many detectable labels can be purchased or synthesized that already contain end groups that facilitate the coupling of the detectable label to the capture moiety or the detection moiety, such as, N10-(3-sulfopropyl)-N-(3-carboxypropyl)-acridinium-9-carboxamide, otherwise known as CPSP-Acridinium Ester or N10-(3-sulfopropyl)-N-(3-sulfopropyl)-acridinium-9-carboxamide, otherwise known as SPSP-Acridinium Ester.

The capture antigen-target antibody complex or the capture antigen-target antibody-detection moiety complex can be, but does not have to be, separated from the remainder of the test sample prior to quantification of the label. For example, if the capture moiety (e.g. capture antigen) is bound to a solid support or solid phase, such as, but not limited to, a well of a reaction tray, a bead or a microparticle, separation can be accomplished by removing the fluid (of the test sample) from contact with the solid support. In some embodiments, if the capture moiety (e.g. capture antigen) is bound to a solid support it can be simultaneously contacted with the sample and the detection moiety, followed by removal of the fluid (test sample) from contact with the solid support. If the capture antigen is not bound to a solid support, then the capture antigen-target antibody complex or the capture antigen-target antibody-detection moiety complex does not have to be removed from the test sample for quantification of the amount of the label.

After formation of the labeled capture antibody-target antibody complex or the capture antibody-target antibody-labeled detection moiety complex, the amount of label in the complex is quantified using techniques known in the art. For example, if an enzymatic label is used, the labeled complex is reacted with a substrate for the label that gives a quantifiable reaction such as the development of color. If the label is a radioactive label, the label is quantified using a scintillation counter. If the label is a fluorescent label, the label is quantified by stimulating the label with a light of one color (which is known as the “excitation wavelength”) and detecting another color (which is known as the “emission wavelength”) that is emitted by the label in response to the stimulation. If the label is a chemiluminescent label, the label is quantified detecting the light emitted either visually or by using luminometers, x-ray film, high speed photographic film, a CCD camera, etc. Once the amount of the label in the complex has been quantified, the concentration of the marker (e.g. the target antibody) in the test sample is determined by use of a standard curve that has been generated using serial dilutions of the marker of known concentration. Other than using serial dilutions of the marker, the standard curve can be generated gravimetrically, by mass spectroscopy and by other techniques known in the art.

The methods and kits as described herein encompass other reagents and methods for carrying out the immunoassay. For instance, encompassed are various buffers such as are known in the art and/or which can be readily prepared or optimized to be employed, e.g., for washing, as a conjugate diluent, and/or as a calibrator diluent. An exemplary conjugate diluent is an ARCHITECT® or ALINITY® diluent (Abbott Laboratories, Abbott Park, IL) containing one or more of 2-(N-morpholino) ethanesulfonic acid (MES), another salt, protein blockers, an antimicrobial and detergent. An exemplary calibrator diluent is ARCHITECT® or ALINITY® calibrator diluent (Abbott Laboratories, Abbott Park, IL), which comprises one or more of a buffer containing MES, another salt, a protein blocker and an antimicrobial.

Furthermore, as previously mentioned, the methods and kits optionally are adapted for use on an automated or semi-automated system. Some of the differences between an automated or semi-automated system as compared to a non-automated system (e.g., ELISA) include the substrate to which the capture moiety is attached (which can impact sandwich formation and analyte reactivity), and the length and timing of the capture, detection and/or any optional wash steps. Whereas a non-automated format such as an ELISA may include a relatively longer incubation time with sample and capture reagent (e.g., about 2 hours) an automated or semi-automated format (e.g., ARCHITECT® or ALINITY®) may have a relatively shorter incubation time (e.g., approximately 18 minutes for ARCHITECT® or ALINITY®). Similarly, whereas a non-automated format such as an ELISA may incubate a detection antibody such as the conjugate reagent (Pb264) for a relatively longer incubation time (e.g., about 2 hours), an automated or semi-automated format (e.g., ARCHITECT® or ALINITY®) may have a relatively shorter incubation time (e.g., approximately 4 minutes for the ARCHITECT® or ALINITY®).

### 3. Methods for Detecting and/or Diagnosing Orthopoxvirus Infection in a Subject

In some aspects, provided herein are methods for detecting orthopoxvirus infection in a subject and/or determining orthopoxvirus vaccination status in a subject. In some aspects, provided herein are methods for detecting orthopoxvirus infection in a subject. In some aspects, provided herein are methods for determining orthopoxvirus vaccination status in a subject. In some aspects, provided herein are methods of differentiating between an orthopoxvirus infection and previous orthopoxvirus vaccination in a subject. As used herein, the term “orthopoxvirus vaccination status” refers to determining whether a subject has or has not received a vaccine against one or more orthopoxviruses. For example, a vaccine commonly used against smallpox virus and other orthopoxviruses comprises a dosage of vaccinia virus. Accordingly, determining “orthopoxvirus vaccination status” in some embodiments refers to determining whether a subject has received a dose of vaccinia virus. The vaccinia virus is antigenically highly similar to the smallpox virus and is thus widely used as a vaccine against smallpox. Vaccination with vaccinia virus also confers immunity against other virus from the orthopoxvirus genus.

The methods provided herein comprise performing an immunoassay as described above. In some embodiments, the immunoassay comprises determining the presence or amount of the target antibody in the sample based upon the detectable signal generated upon formation of the capture antigen-target antibody complex (e.g. in a direct immunoassay) or based upon the detectable signal generated upon formation of the capture antigen-target antibody-detection moiety complex (e.g. in an indirect immunoassay). In some embodiments, the method further comprises determining the amount of the target antibody in the sample based upon the detectable signal generated.

In some embodiments, the method further comprises determining that the subject is infected with an orthopoxvirus (e.g. monkeypox) when the amount of the target antibody (e.g. the level of the detectable signal, which is indicative of the amount of target antibody) is equal to or above a reference value. In some embodiments, the method further comprises determining that the subject has received a vaccine against orthopoxvirus (e.g. a smallpox vaccine, an injection with vaccinia virus) when the amount of the target antibody (e.g. the level of the detectable signal, which is indicative of the amount of target antibody) is equal to or above a reference value. In some embodiments, the method further comprises determining that the subject is not infected with an orthopoxvirus (e.g. monkeypox) when the amount of the target antibody (e.g. the level of the detectable signal, which is indicative of the amount of target antibody) is below a reference value. In some embodiments, the method further comprises determining that the subject has not received a vaccine against orthopoxvirus when the amount of the target antibody (e.g. the level of the detectable signal, which is indicative of the amount of target antibody) is below a reference value.

In some aspects, provided herein are methods for detecting orthopoxvirus infection in a subject and/or determining orthopoxvirus vaccination status in a subject. In some embodiments, the methods comprise performing an immunoassay as described herein, including an immunoassay described above in section 2.

In some embodiments, the immunoassay comprises contacting a sample obtained from a subject with a capture antigen. In some embodiments, the capture antigen comprises a recombinant A33R protein. In some embodiments, the recombinant A33R protein has at least 80% sequence identity to SEQ ID NO: 1. In some embodiments, the capture antigen is a recombinant A33R protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 1.

In some embodiments, the immunoassay comprises contacting a sample obtained from a subject with a capture antigen. In some embodiments, the capture antigen comprises a recombinant A35R protein. In some embodiments, the recombinant A35R protein has at least 80% sequence identity to SEQ ID NO: 2. In some embodiments, the capture antigen is a recombinant A35R protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 2.

In some embodiments, the immunoassay comprises contacting a sample obtained from a subject with a capture antigen. In some embodiments, the capture antigen comprises a recombinant A27L protein. In some embodiments, the recombinant A27L protein has at least 80% sequence identity to SEQ ID NO: 3. In some embodiments, the capture antigen is a recombinant A27L protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 3.

In some embodiments, the immunoassay comprises contacting a sample obtained from a subject with a capture antigen. In some embodiments, the capture antigen comprises a recombinant BR6 protein. In some embodiments, the recombinant BR6 protein has at least 80% sequence identity to SEQ ID NO: 4. In some embodiments, the capture antigen is a recombinant BR6 protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 4.

In some embodiments, the immunoassay comprises contacting a sample obtained from a subject with a capture antigen. In some embodiments, the capture antigen comprises a recombinant H3L protein. In some embodiments, the recombinant H3L protein has at least 80% sequence identity to SEQ ID NO: 5. In some embodiments, the capture antigen is a recombinant H3L protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 5.

In some embodiments, the immunoassay further comprises contacting the sample with a detection moiety. In some embodiments, the detection moiety comprises an anti-human IgM antibody. An immunoassay comprising contacting the sample with a recombinant A33R protein as the capture antigen (e.g. a recombinant A33R protein having at least at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 1, a recombinant A35R protein having at least at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 2, a recombinant A27L protein having at least at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 3, a recombinant BR6 protein having at least at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 4, or a recombinant H3L protein having at least at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 5) and an anti-human IgM antibody as the detection moiety is referred to herein as the “Orthopox IgM assay” or the “Orthopox IgM immunoassay”. In some embodiments, contacting the sample with the capture antigen (e.g. the recombinant A33R, A35R, A27L, B6R, or H3L protein) and the detection moiety (e.g. the anti-human IgM antibody) results in the formation of a capture antigen-target antibody-detection moiety complex, which complex produces a detectable signal. In some embodiments, the immunoassay further comprises determining the presence or amount of the target antibody (e.g. the antibody against A33R, A35R, A27L, B6R, or H3L) in the sample based upon the detectable signal. In some embodiments, the method further comprises determining that the subject is infected with an orthopoxvirus (e.g. monkeypox) and/or has received a vaccination against one or more orthopoxviruses (e.g. has received a vaccinia virus) when the amount of the target antibody (e.g. the level of the detectable signal, which is indicative of the amount of target antibody) is equal to or above a reference value. In some embodiments, the method further comprises determining that the subject is not infected with an orthopoxvirus (e.g. monkeypox) and/or has not received a vaccination against one or more orthopoxviruses (e.g. has not received a vaccinia virus) when the amount of the target antibody (e.g. the level of the detectable signal, which is indicative of the amount of target antibody) is below a reference value.

In some embodiments, the Orthopox IgM immunoassay displays at least 75% sensitivity (e.g. sensitivity in accurately diagnosing monkeypox infection in a subject, sensitivity in accurately determining that a subject has received an orthopoxvirus vaccine). In some embodiments, the Orthopox IgM immunoassay displays at least 85% sensitivity. In some embodiments, the Orthopox IgM immunoassay displays at least 90% sensitivity. In some embodiments, the Orthopox IgM immunoassay displays at least 95% sensitivity. In some embodiments, the Orthopox IgM immunoassay displays at least 99% sensitivity.

In some embodiments, the Orthopox IgM immunoassay displays at least 75% specificity (e.g. specificity in accurately differentiating subjects not having monkeypox infection from those having monkeypox infection, specificity in accurately differentiating subjects not having received a vaccinia vaccine from those that have received a vaccine). In some embodiments, the Orthopox IgM immunoassay displays at least 85% specificity. In some embodiments, the Orthopox IgM immunoassay displays at least 90% specificity. In some embodiments, the Orthopox IgM immunoassay displays at least 95% specificity.

In some embodiments, the Orthopox IgM immunoassay displays at least 75% sensitivity and at least 75% specificity. In some embodiments, the Orthopox IgM immunoassay displays at least 85% sensitivity and at least 75% specificity. In some embodiments, the Orthopox IgM immunoassay displays at least 95% sensitivity and at least 75% specificity. In some embodiments, the Orthopox IgM immunoassay displays at least 99% sensitivity and at least 75% specificity.

In some embodiments, the Orthopox IgM immunoassay displays at least 85% sensitivity and at least 85% specificity. In some embodiments, the Orthopox IgM immunoassay displays at least 90% sensitivity and at least 85% specificity. In some embodiments, the Orthopox IgM immunoassay displays at least 95% sensitivity and at least 85% specificity. In some embodiments, the Orthopox IgM immunoassay displays at least 99% sensitivity and at least 85% specificity.

In some embodiments, the Orthopox IgM immunoassay displays at least 90% sensitivity and at least 90% specificity. In some embodiments, the Orthopox IgM immunoassay displays at least 95% sensitivity and at least 90% specificity. In some embodiments, the Orthopox IgM immunoassay displays at least 99% sensitivity and at least 90% specificity.

In some embodiments, the Orthopox IgM immunoassay displays at least 95% sensitivity and at least 95% specificity. In some embodiments, the Orthopox IgM immunoassay displays at least 99% sensitivity and at least 95% specificity.

In some embodiments, the detection moiety comprises an anti-human IgG antibody. In some embodiments, the capture antigen comprises a recombinant A33R, A35R, A27L, B6R, or H3L protein (e.g. a recombinant A33R protein having at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 1, a recombinant A35R protein having at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 2, a recombinant A27L protein having at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 3, a recombinant B6R protein having at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 4, or a recombinant H3L protein having at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 5) and the detection moiety comprises an anti-human IgG antibody. An immunoassay comprising contacting the sample with a recombinant A33R A35R, A27L, B6R, or H3L protein as the capture antigen (e.g. a recombinant A33R protein having at least at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 1, a recombinant A35R protein having at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 2, a recombinant A27L protein having at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 3, a recombinant B6R protein having at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 4, or a recombinant H3L protein having at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 5) and an anti-human IgG antibody as the detection moiety is referred to herein as the “Orthopox IgG assay” or the “Orthopox IgG immunoassay”.

In some embodiments, the Orthopox IgG assay displays at least 75% sensitivity (e.g. sensitivity in accurately diagnosing monkeypox infection in a subject, sensitivity in accurately determining that a subject has received an orthopoxvirus vaccine). In some embodiments, the Orthopox IgG immunoassay displays at least 85% sensitivity. In some embodiments, the Orthopox IgG immunoassay displays at least 90% sensitivity. In some embodiments, the Orthopox IgG immunoassay displays at least 95% sensitivity.

In some embodiments, the Orthopox IgG immunoassay displays at least 75% specificity (e.g. specificity in accurately differentiating subjects not having monkeypox infection from those having monkeypox infection, specificity in accurately differentiating subjects not having received a vaccinia vaccine from those that have received a vaccine). In some embodiments, the Orthopox IgG immunoassay displays at least 85% specificity. In some embodiments, the Orthopox IgG immunoassay displays at least 90% specificity. In some embodiments, the Orthopox IgG immunoassay displays at least 95% specificity. In some embodiments, the Orthopox IgG immunoassay displays at least 99% specificity.

In some embodiments, the Orthopox IgG immunoassay displays at least 75% specificity and at least 75% sensitivity. In some embodiments, the Orthopox IgG immunoassay displays at least 85% sensitivity and at least 75% specificity. In some embodiments, the Orthopox IgG immunoassay displays at least 95% sensitivity and at least 75% specificity.

In some embodiments, the Orthopox IgG immunoassay displays at least 85% sensitivity and at least 85% specificity. In some embodiments, the Orthopox IgG immunoassay displays at least 90% sensitivity and at least 85% specificity. In some embodiments, the Orthopox IgG immunoassay displays at least 95% sensitivity and at least 85% specificity.

In some embodiments, the Orthopox IgG immunoassay displays at least 85% sensitivity and at least 90% specificity. In some embodiments, the Orthopox IgG immunoassay displays at least 85% sensitivity and at least 95% specificity. In some embodiments, the Orthopox IgG immunoassay displays at least 85% sensitivity and at least 99% specificity.

In some embodiments, the Orthopox IgG immunoassay displays at least 90% sensitivity and at least 90% specificity. In some embodiments, the Orthopox IgG immunoassay displays at least 95% sensitivity and at least 90% specificity. In some embodiments, the Orthopox IgG immunoassay displays at least 90% sensitivity and at least 95% specificity. In some embodiments, the Orthopox IgG immunoassay displays at least 90% sensitivity and at least 99% specificity.

In some embodiments, the Orthopox IgG immunoassay displays at least 95% sensitivity and at least 95% specificity. In some embodiments, the Orthopox IgG immunoassay displays at least 95% sensitivity and at least 99% specificity. In some embodiments, the method for detecting orthopoxvirus infection in a subject and/or determining orthopoxvirus vaccination status in a subject comprises performing a direct immunoassay involving contacting a sample obtained from a subject with a capture antigen. In some embodiments, the capture antigen comprises a recombinant A33R, A35R, A27L, B6R, or H3L protein. In some embodiments, the recombinant A33R protein has at least 80% sequence identity to SEQ ID NO: 1. In some embodiments, the capture antigen is a recombinant A33R protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 1. In some embodiments, the immunoassay is a direct immunoassay wherein the capture antigen (e.g. the recombinant A33R protein having at least 80% sequence identity to SEQ ID NO: 1) is also detectably labeled. In such an immunoassay, the recombinant A33R protein serves as both the capture antigen and the detection antigen. In some embodiments, the recombinant A35R protein has at least 80% sequence identity to SEQ ID NO: 2. In some embodiments, the capture antigen is a recombinant A35R protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 2. In some embodiments, the immunoassay is a direct immunoassay wherein the capture antigen (e.g. the recombinant A35R protein having at least 80% sequence identity to SEQ ID NO: 2) is also detectably labeled. In such an immunoassay, the recombinant A35R protein serves as both the capture antigen and the detection antigen. In some embodiments, the recombinant A27L protein has at least 80% sequence identity to SEQ ID NO: 3. In some embodiments, the capture antigen is a recombinant A27L protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 3. In some embodiments, the immunoassay is a direct immunoassay wherein the capture antigen (e.g. the recombinant A27L protein having at least 80% sequence identity to SEQ ID NO: 3) is also detectably labeled. In such an immunoassay, the recombinant A27L protein serves as both the capture antigen and the detection antigen. In some embodiments, the recombinant B6R protein has at least 80% sequence identity to SEQ ID NO: 4. In some embodiments, the capture antigen is a recombinant B6R protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 4. In some embodiments, the immunoassay is a direct immunoassay wherein the capture antigen (e.g. the recombinant B6R protein having at least 80% sequence identity to SEQ ID NO: 4) is also detectably labeled. In such an immunoassay, the recombinant B6R protein serves as both the capture antigen and the detection antigen. In some embodiments, the recombinant H3L protein has at least 80% sequence identity to SEQ ID NO: 5. In some embodiments, the capture antigen is a recombinant H3L protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 5. In some embodiments, the immunoassay is a direct immunoassay wherein the capture antigen (e.g. the recombinant H3L protein having at least 80% sequence identity to SEQ ID NO: 5) is also detectably labeled. In such an immunoassay, the recombinant H3L protein serves as both the capture antigen and the detection antigen.

A direct immunoassay comprising contacting the sample with a recombinant A33R protein as the capture antigen (e.g. a recombinant A33R protein having at least at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 1, a recombinant A35R protein having at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 2, a recombinant A27L protein having at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 3, a recombinant B6R protein having at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 4, or a recombinant H3L protein having at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity to SEQ ID NO: 5, which also serves as the detection moiety) is referred to herein as the “Orthopox Total Ig assay” or the “Orthopox Total Ig immunoassay”.

In some embodiments, the Orthopox Total Ig immunoassay displays at least 65% sensitivity (e.g. sensitivity in accurately diagnosing monkeypox infection in a subject, sensitivity in accurately determining that a subject has received an orthopoxvirus vaccine). In some embodiments, the Orthopox Total Ig immunoassay displays at least 70% sensitivity. In some embodiments, the Orthopox Total Ig immunoassay displays at least 75% sensitivity. In some embodiments, the Orthopox Total Ig immunoassay displays at least 80% sensitivity. In some embodiments, the Orthopox Total Ig immunoassay displays at least 85% sensitivity. In some embodiments, the Orthopox Total Ig immunoassay displays at least 90% sensitivity. In some embodiments, the Orthopox Total Ig immunoassay displays at least 95% sensitivity.

In some embodiments, the Orthopox Total Ig immunoassay displays at least 75% specificity (e.g. specificity in accurately differentiating subjects not having monkeypox infection from those having monkeypox infection, specificity in accurately differentiating subjects not having received a vaccinia vaccine from those that have received a vaccine). In some embodiments, the Orthopox Total Ig immunoassay displays at least 85% specificity. In some embodiments, the Orthopox Total Ig immunoassay displays at least 90% specificity. In some embodiments, the Orthopox Total Ig immunoassay displays at least 95% specificity. In some embodiments, the Orthopox Total Ig immunoassay displays at least 99% specificity.

In some embodiments, the Orthopox Total Ig immunoassay displays at least 65% sensitivity and at least 75% specificity. In some embodiments, the Orthopox Total Ig immunoassay displays at least 65% sensitivity and at least 85% specificity. In some embodiments, the Orthopox Total Ig immunoassay displays at least 65% sensitivity and at least 90% specificity. In some embodiments, the Orthopox Total Ig immunoassay displays at least 65% sensitivity and at least 95% specificity. In some embodiments, the Orthopox Total Ig immunoassay displays at least 65% sensitivity and at least 99% specificity.

In some embodiments, the Orthopox Total Ig immunoassay displays at least 75% sensitivity and at least 75% specificity. In some embodiments, the Orthopox Total Ig immunoassay displays at least 75% sensitivity and at least 85% specificity. In some embodiments, the Orthopox Total Ig immunoassay displays at least 75% sensitivity and at least 90% specificity. In some embodiments, the Orthopox Total Ig immunoassay displays at least 75% sensitivity and at least 95% specificity. In some embodiments, the Orthopox Total Ig immunoassay displays at least 75% sensitivity and at least 99% specificity.

In some embodiments, the Orthopox Total Ig immunoassay displays at least 80% sensitivity and at least 80% specificity. In some embodiments, the Orthopox Total Ig immunoassay displays at least 80% sensitivity and at least 85% specificity. In some embodiments, the Orthopox Total Ig immunoassay displays at least 80% sensitivity and at least 90% specificity. In some embodiments, the Orthopox Total Ig immunoassay displays at least 80% sensitivity and at least 95% specificity. In some embodiments, the Orthopox Total Ig immunoassay displays at least 80% sensitivity and at least 99% specificity.

In some embodiments, provided herein is a method of differentiating between an orthopoxvirus infection and previous orthopoxvirus vaccination in a subject. In some embodiments, the method comprises performing a competitive immunoassay that that measures the avidity of antibodies in a sample, as described in section 2. In some embodiments, the competitive immunoassay is referred to as an “IgG Avidity assay” or an “IgG Avidity immunoassay”. For example, in some embodiments the immunoassay is a competitive immunoassay that measures the avidity of anti-A33R, anti-A35R, anti-A27L, anti-B6R or anti-H3L antibodies in a sample. In some embodiments, the immunoassay (e.g. the competitive immunoassay) uses a recombinant A33R protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 1, uses a recombinant A35R protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 2, uses a recombinant A27L protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 3, uses a recombinant BR6 protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 4 or uses a recombinant H3L protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 5, as the capture antigen to form the solid phase antigen and the liquid phase antigen. In some embodiments, the competitive immunoassay uses an anti-human IgG antibody as a detection moiety.

In some embodiments, the competitive immunoassay comprises two assays, a first assay involving contacting a sample with a solid phase antigen and a second assay involving contacting a sample with a solid phase and liquid phase antigen. In some embodiments, the results of the two assays are compared in order to generate an avidity index. In some embodiments, the first assay comprises contacting the sample with a solution comprising a solid phase antigen. In some embodiments, the solid phase antigen comprises a recombinant A33R protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 1 attached to a solid phase (e.g. a microparticle). In some embodiments, the solid phase antigen comprises a recombinant A35R protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 2 attached to a solid phase (e.g. a microparticle). In some embodiments, the solid phase antigen comprises a recombinant A27L protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 3 attached to a solid phase (e.g. a microparticle). In some embodiments, the solid phase antigen comprises a recombinant B6R protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 4 attached to a solid phase (e.g. a microparticle). In some embodiments, the solid phase antigen comprises a recombinant H3L protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NO: 5 attached to a solid phase (e.g. a microparticle). In some embodiments, the solution does not contain a liquid phase antigen (e.g. does not contain a recombinant A33R, A35R, A27L, B6R, or H3L protein in solution). Accordingly, in the first assay, both low avidity and high avidity antibodies (e.g. low avidity and high avidity anti-A33R, anti-A35R, anti-A27L, anti-B6R, or anti-H3L antibodies) will bind to the solid phase antigen, thereby producing solid phase antigen-low avidity antibody complexes and solid phase antigen-high avidity antibody complexes in the sample. In some embodiments, following incubation of the sample with the solution comprising a solid phase antigen a detection moiety is added, which binds to the antibodies bound to the solid phase antigen. As described herein, in some embodiments the detection moiety comprises a detectable label. The detection moiety binds to the capture antigen-target antibody complex, thereby producing a detectable signal. In this first assay, the amount of detectable signal is a reflection of the amount of both low avidity antibodies (e.g. low avidity anti-A33R, anti-A35R, anti-A27L, anti-B6R or anti-H3L antibodies) and high avidity antibodies (e.g. high avidity anti-A33R, anti-A35R, anti-A27L, anti-B6R or anti-H3L antibodies) in the sample.

In some embodiments, the second assay comprises contacting the sample with a solution comprising a liquid phase antigen (e.g. a recombinant A33R, A35R, A27L, B6R, or H3L protein in solution) and a solid phase antigen (e.g. a recombinant A33R, A35R, A27L, B6R or H3L protein attached to a solid phase). For example, in some embodiments the second assay comprises contacting the sample with a solution comprising a recombinant A33R, A35R, A27L, B6R or H3L protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NOS: 1, 2, 3, 4 or 5, respectively attached to a solid phase (i.e. a solid phase antigen) and a solution comprising a recombinant A33R, A35R, A27L, B6R or H3L protein having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% identity) to SEQ ID NOS: 1, 2, 3, 4 or 5, respectively, in solution (i.e. a liquid phase antigen). In the second assay, low avidity antibodies (e.g. low avidity anti-A33R, anti-A35R, anti-A27L, anti-B6R or anti-H3L antibodies) preferentially bind to the solid phase antigen, thereby producing solid phase antigen-low avidity antibody complexes, whereas high avidity antibodies (e.g. high avidity anti-A33R, anti-A27L, anti-B6R or anti-H3L antibodies) preferentially bind to the liquid phase antigen, thereby producing liquid phase antigen-high avidity antibody complexes. In some embodiments, the second assay comprises a wash step, which removes antibodies not bound to the solid phase antigen (e.g. removes the liquid phase antigen-high avidity antibody complexes). In some embodiments, following the wash step a detection moiety is added, which binds to the antibodies bound to the solid phase antigen (e.g. binds to the solid phase antigen-low avidity antibody complexes). As described herein, in some embodiments the detection moiety comprises a detectable label. The detection moiety binds to the capture antigen-target antibody complex (e.g. the solid phase antigen-low avidity antibody complexes), thereby producing a detectable signal. In this second assay, the amount of detectable signal is a reflection of the amount of low avidity antibodies (e.g. low avidity anti-A33R, anti-A27L, anti-B6R or anti-H3L antibodies) in the sample.

In some embodiments, an avidity index of the sample is calculated by mathematically transforming the results of the first and second assay as follows:

$$Avidity index = [ 1 - ( signal assay \#2 / signal assay \#1 ) ] \times 100.$$

As mentioned previously herein, the avidity index is indicative of the amount of high-avidity anti-A33R, anti-A35R, anti-A27L, anti-B6R, or anti-H3L antibodies in a sample obtained from a subject. In some aspects, as shown in the above formula, the avidity index is determined by first determining the ratio of signal generated by the detectable label in the second assay (assay #2) to the signal generated by the detectable label in a first assay (assay #1). Once the ratio is determined, then it is subtracted from 1 and then multiplied by 100.

The avidity index is proportional to the amount of high avidity antibodies present in the sample, with a lower avidity index indicating fewer high avidity antibodies present in the sample and a higher avidity index indicating greater numbers of high avidity antibodies present in the sample. Accordingly, in some embodiments a high avidity index is indicative of a previous vaccination against orthopoxvirus, which generates increasing numbers of high avidity antibodies over time. In contrast, in some embodiments a low avidity index is indicative of current or recent orthopoxvirus infection, which generates low avidity antibodies in response to the infection.

In some embodiments, the IgG Avidity immunoassay differentiates between recent/current orthopoxvirus infection and past orthopoxvirus infection or vaccination against one or more orthopoxviruses based upon the avidity index of a sample. In some embodiments, the method comprises determining that the subject is recently infected (within the last one month, within the last two months, within the last three months, within the last four months, within the last five months, within the last six months, within the last 1-6 months, 1-5 months, 1-4 months, 1-3 months, 1-2 months, 2-6 months, 2-5 months, 2-4 months, 2-3 months, 3-6 months, 3-4 months, or 4-5 months) with orthopoxvirus (e.g. monkeypox) or recently vaccinated (within the last one month, within the last two months, within the last three months, within the last four months, within the last five months, within the last six months, within the last 1-6 months, 1-5 months, 1-4 months, 1-3 months, 1-2 months, 2-6 months, 2-5 months, 2-4 months, 2-3 months, 3-6 months, 3-4 months, or 4-5 months) against orthopoxvirus (e.g. monkeypox) if the avidity index of the sample is less than 45% and determining that the subject has received a vaccination against one or more orthopoxviruses if the avidity index of the sample is equal to or greater than 45%. In some embodiments, the method comprises determining that the subject is recently infected with orthopoxvirus (e.g. monkeypox) or recently vaccinated against orthopoxvirus (e.g., monkeypox) if the avidity index of the sample is less than 45%, less than 44%, less than 43%, less than 42%, less than 41%, less than 40%, less than 39%, less than 38%, less than 37%, less than 36%, less than 35%, less than 34%, less than 33%, less than 32%, less than 31%, or less than 30%. In some embodiments, the method comprises determining that the subject has previously received (namely, within the last 3 months, within the last 4 months, within the last 5 months, within the last 6 months, within the last 7 months, within the last 8 months, within the last 9 months, within the last 10 months, within the last 11 months, within the last 12 months, within the last 3-12 months, 3-11 months, 3-10 months, 3-9 months, 3-8 months, 3-7 months, 3-6 months, 3-5 months, 3-4 months, 4-12 months, 4-11 months, 4-10 months, 4-9 months, 4-8 months, 4-7 months, 4-6 months, 4-5 months, 5-12 months, 5-11 months, 5-10 months, 5-9 months, 5-8 months, 5-7 months, 5-6 months, 6-12 months, 6-11 months, 6-10 months, 6-9 months, 6-8 months, 6-7 months, or 3-6 months) a vaccination against one or more orthopoxviruses if the avidity index of the sample is greater than 45%, greater than 46%, greater than 47%, greater than 48%, greater than 49%, greater than 50%, greater than 51%, greater than 52%, greater than 53%, greater than 54%, greater than 55%, greater than 56%, greater than 57%, greater than 58%, greater than 59%, or greater than 60%.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. A ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative. The immunoassays described herein may show an ROC of at least 0.6, at least about 0.7, at least about 0.8, or at least about 0.9.

In some embodiments, the methods provided herein further comprise treating the subject for an orthopoxvirus infection. Suitable treatments for orthopoxvirus infection include, for example, antiviral medications such as tecovirimat, brincidofovir, and vaccinia immune globulin.

### 4. Apparatus, Non-Transitory Machine-Readable Storage Medium, and Systems for Detecting and/or Diagnosing Orthopoxvirus Infection in a Subject

In some some embodiments, disclosed herein are apparatus, machine-readable storage mediums and systems for use in implementing or performing the immunoassays and/or methods described in Sections 2 and/or 3. More specifically, the apparatus, machine-readable storage mediums, and systems described herein can be used for detecting orthopoxvirus infection in a subject and/or determining orthopoxvirus vaccination status in a subject and communicate from an apparatus (e.g., such as a point-of-care, non-point of care, or a point-of-care and non-point-of-care apparatus), the presence or absence of orthopoxvirus infection or the orthopoxvirus vaccination status of a subject. The term “apparatus” is used interchangeably herein with “device” or “instrument”.

In some aspects, provided herein are apparatus, machine-readable storage mediums and systems for detecting orthopoxvirus infection in a subject. In some aspects, provided herein are apparatus, machine-readable storage mediums and systems for determining orthopoxvirus vaccination status in a subject. In some aspects, provided herein are apparatus, machine-readable storage mediums and systems of differentiating between an orthopoxvirus infection and previous orthopoxvirus vaccination in a subject. As used herein, the term “orthopoxvirus vaccination status” refers to determining whether a subject has or has not received a vaccine against one or more orthopoxviruses. For example, a vaccine commonly used against smallpox virus and other orthopoxviruses comprises a dosage of vaccinia virus. Accordingly, determining “orthopoxvirus vaccination status” in some embodiments refers to determining whether a subject has received a dose of vaccinia virus. The vaccinia virus is antigenically highly similar to the smallpox virus and is thus widely used as a vaccine against smallpox. Vaccination with vaccinia virus also confers immunity against other virus from the orthopoxvirus genus.

In another embodiment, the present disclosure relates to an apparatus, device, or instrument. The apparatus, device, or instrument contains software to execute one or more tasks, including the performance of the immunoassays and/or methods described in Sections 2 and/or 3. In some embodiments, the apparatus, device, or instrument contains software to automatically determine the next appropriate step in the methods described herein. For example, the apparatus, device, or instrument may contain software that determines the presence or absence of orthopoxvirus infection in a subject or the orthopoxvirus vaccination status of a subject. The software may display this determination, such as on a graphical user interface.

In some embodiments, the apparatus, device, or instruments stores software that instructs processor or processor circuitry to execute or instantiate a given task. In some embodiments, the software stores machine-readable instructions that cause processor circuitry to execute or instantiate a given task. The machine-readable instructions may be one or more executable programs or portion(s) of an executable program for execution by a computer. The programs may be embodied in software stored on a non-transitory computer readable storage medium such as a CD-ROM, a floppy disk, a hard drive, a DVD, a Blu-ray disk, or a memory associated with the processors. Alternatively, the entire programs and/or parts thereof could alternatively be executed by a device other than the processors and/or embodied in firmware or dedicated hardware. Additionally or alternatively, processes may be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

The machine-readable instructions may be stored in one or more of a compressed format, an encrypted format, a fragmented format, a compiled format, an executable format, a packaged format, etc. Machine readable instructions as described herein may be stored as data (e.g., portions of instructions, code, representations of code, etc.) that may be utilized to create, manufacture, and/or produce machine executable instructions. For example, the machine-readable instructions may be fragmented and stored on one or more storage devices and/or computing devices (e.g., servers). The machine-readable instructions may require one or more of installation, modification, adaptation, updating, combining, supplementing, configuring, decryption, decompression, unpacking, distribution, reassignment, compilation, etc. in order to make them directly readable, interpretable, and/or executable by a computing device and/or other machine. For example, the machine-readable instructions may be stored in multiple parts, which are individually compressed, encrypted, and stored on separate computing devices, wherein the parts when decrypted, decompressed, and combined form a set of executable instructions that implement a program such as that described herein.

In another example, the machine-readable instructions may be stored in a state in which they may be read by a computer, but require addition of a library (e.g., a dynamic link library (DLL)), a software development kit (SDK), an application programming interface (API), etc. in order to execute the instructions on a particular computing device or other device. In another example, the machine-readable instructions may need to be configured (e.g., settings stored, data input, network addresses recorded, etc.) before the machine-readable instructions and/or the corresponding program(s) can be executed in whole or in part. Thus, the disclosed machine-readable instructions and/or corresponding program(s) are intended to encompass such machine-readable instructions and/or program(s) regardless of the particular format or state of the machine-readable instructions and/or program(s) when stored or otherwise at rest or in transit.

The machine-readable instructions described herein can be represented by any past, present, or future instruction language, scripting language, programming language, etc. For example, the machine-readable instructions may be represented using any of the following languages: C, C++, Java, C#, Perl, Python, JavaScript, HyperText Markup Language (HTML), Structured Query Language (SQL), Swift, etc.

The machine-readable instructions may be stored on a non-transitory computer and/or non-transitory machine-readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term “non-transitory computer readable medium” is defined to include any type of computer readable storage device and/or storage disk and to exclude propagating signals and to exclude transmission media.

In some further aspects, disclosed herein is a system comprises: (a) a sample receiving component that is configured to receive a sample from a subject; (b) a capture antigen comprising at least one detectable label configured to make contact with the sample to form a capture antigen-target antibody, complex, wherein the capture antigen comprises a recombinant A33R, A35R, A27L, B6R, or H3L protein; (c) a detection component configured to measure a signal generated by the detectable label in the capture antigen-target antibody complex; and (d) an output component that indicates an amount of target antibody in the sample based on the signal.

In yet a further aspect, the system comprises: (a) a sample receiving component configured to receive a sample from a subject; (b) a sample analysis component configured to determine a total amount of anti-A33R, anti-A35R, anti-A27L, anti-B6R, or anti-H3L antibodies in the sample and an amount of low avidity anti-A33R, anti-A35R, anti-A27L, anti-B6R, or anti-H3L antibodies in the sample to arrive at an index representing a relationship between the total amount of anti-A33R, anti-A35R, anti-A27L, anti-B6R, or anti-H3L antibodies and the amount of low avidity anti-A33R, anti-A35R, anti-A27L, anti-B6R, or anti-H3L antibodies; and (c) an output component that indicates: 1) the total amount of anti-A33R, anti-A35R, anti-A27L, anti-B6R, or anti-H3L antibodies and the amount of low avidity anti-A33R, anti-A35R, anti-A27L, anti-B6R, or anti-H3L antibodies, 2) the index, 3) whether the index is above or below a preset threshold, and/or 4) whether the subject is likely infected with an orthopoxvirus or has received a vaccination against one or more orthopoxviruses.

In yet still a further aspect, the system comprises: (a) a sample receiving component configured to receive a sample from a subject; (b) a sample analysis component configured to determine a total amount of anti-A33R, anti-A35R, anti-A27L, anti-B6R, or anti-H3L antibodies in the sample and an amount of low avidity anti-A33R, anti-A35R, anti-A27L, anti-B6R, or anti-H3L antibodies in the sample to arrive at an index representing a relationship between the total amount of anti-A33R, anti-A35R, anti-A27L, anti-B6R, or anti-H3L antibodies and the amount of low avidity anti-A33R, anti-A35R, anti-A27L, anti-B6R, or anti-H3L antibodies; and (c) an output component that indicates: 1) the total amount of anti-A33R, anti-A35R, anti-A27L, anti-B6R, or anti-H3L antibodies and the amount of low avidity anti-A33R, anti-A35R, anti-A27L, anti-B6R, or anti-H3L antibodies, 2) the index, 3) whether the index is above or below a preset threshold, and/or 4) whether the subject is likely infected with an orthopoxvirus or has received a vaccination against one or more orthopoxviruses.

The systems provided herein are capable of performing an immunoassay and/or methods as described previously in Sections 2 and/or 3.

In the above systems, the at least one sample receiving component can comprise at least one test or sample collection container (e.g., a collection tube) or device (e.g., a cartridge). Any device that is capable of receiving a sample is suitable for use in the systems described herein. In some aspects, the sample receiving component is capable of being inserted into a high throughput analyzer, such as the ARCHITECT® i2000 automated immunoassay platform (Abbott Laboratories), or a point-of-care device, such as the i-STAT® device of Abbott Laboratories (Abbott Park, IL).

In the above systems, the detection component can comprise one or more detectors or other detection devices capable of detecting the presence of or measuring the amount of signal generated from the detectable label. For example, the one or more detectors or detection devices can be a luminometer, CCD cameras, video input module cameras, Streak cameras, bolometer, a photodiode, a photodiode array, avalanche photodiodes, photomultipliers, or any combinations thereof can be used. In some aspects, the detection component is contained within a high throughput analyzer, such as the ARCHITECT® i2000 automated immunoassay platform (Abbott Laboratories), or a point-of-care device, such as the i-STAT® device of Abbott Laboratories (Abbott Park, IL). The detection of the presence of or measuring the amount of a signal generated from the detectable label as a result of the performance of the assays described in Sections 2 and/or 3 is herein referred to as a “result”.

In the above systems, the output component conveys, displays, and/or communicates (e.g., reports) the result. In some aspects, the output component displays the result visually (e.g., a readout). In other aspects, the output component conveys the result by means of audio or haptically. In other aspects, the output component conveys, displays and/or communicates the result by a computer, in a document and/or spreadsheet, on a mobile device (e.g., a smart phone), on a website, in an e-mail, or any combination thereof. In some aspects, the output component is contained within a high throughput analyzer, such as the ARCHITECT® i2000 automated immunoassay platform (Abbott Laboratories), or a point-of-care device, such as the i-STAT® device of Abbott Laboratories (Abbott Park, IL).

The immunoassays and methods described in Sections 2 and/or 3 can be implemented or performed in the systems described herein together with a general purpose processor, a Digital Signal Processor (DSP), an Application Specific Integrated Circuit (ASIC), a Field Programmable Gate Array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. The processor can be part of a computing system that also has a user interface port that communicates with a user interface, and which receives commands entered by a user, has at least one memory (e.g., hard drive or other comparable storage, and random access memory) that stores electronic information including a program that operates under control of the processor and with communication via the user interface port, and a video output that produces its output via any kind of video output format, e.g., VGA, DVI, HDMI, DisplayPort, or any other form.

A processor may also be implemented as a combination of computing devices, e.g., a combination of a DSP and a microprocessor, a plurality of microprocessors, one or more microprocessors in conjunction with a DSP core, or any other such configuration. These devices may also be used to select values for devices as described herein. The camera may be a camera based on phototubes, photodiodes, active pixel sensors (CMOS), CCD, photoresistors, photovoltaic cells or other digital image capture technology.

The steps of the immunoassays and methods described in connection with the embodiments disclosed in Sections 2 and/or 3 may be embodied directly in hardware, in a software module executed by a processor, or in a combination of the two. A software module may reside in Random Access Memory (RAM), flash memory, Read Only Memory (ROM), Electrically Programmable ROM (EPROM), Electrically Erasable Programmable ROM (EEPROM), registers, hard disk, a removable disk, a CD-ROM, a cloud, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In one or more example embodiments, the functions described may be implemented in hardware, software, firmware, or any combination thereof. If implemented in software, the functions may be stored on, transmitted over or resulting analysis/calculation data output as one or more instructions, code or other information on a computer-readable medium. Computer-readable media includes both computer storage media and communication media including any medium that facilitates transfer of a computer program from one place to another. A storage media may be any available non-transitory media that can be accessed by a computer. By way of example, such computer-readable media can include RAM, ROM, EEPROM, CD-ROM or other optical disk storage, magnetic disk storage or other magnetic storage devices, or any other medium that can be used to carry or store desired program code in the form of instructions or data structures and that can be accessed by a computer. The memory storage can also be rotating magnetic hard disk drives, optical disk drives, or flash memory based storage drives or other such solid state, magnetic, or optical storage devices. Disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

To the extent the embodiments disclosed herein include or operate in association with memory, storage, and/or computer readable media, then that memory, storage, and/or computer readable media are non-transitory. Accordingly, to the extent that memory, storage, and/or computer readable media are covered by one or more claims, then that memory, storage, and/or computer readable media is only non-transitory.

### 5. Samples

As used herein, “sample”, “test sample”, “biological sample” refer to fluid sample containing or suspected of containing a target antibody (e.g. an antibody produced during orthopoxvirus infection and/or vaccination). In some cases, the sample may be processed prior to performing a method (e.g. an immunoassay) described herein. For example, the sample may be separated or purified from its source prior to analysis. In some embodiments, an unprocessed sample can be assayed directly.

In some embodiments, the sample is a human bodily substance (e.g., bodily fluid, blood such as whole blood, serum, plasma, urine, saliva, sweat, sputum, semen, mucus, lacrimal fluid, lymph fluid, amniotic fluid, interstitial fluid, lung lavage, cerebrospinal fluid, feces, tissue, organ, or the like). In some embodiments, the sample is a whole blood sample, a serum sample, or a plasma sample.

A wide range of volumes of the fluid sample may be analyzed. In a few exemplary embodiments, the sample volume may be about 0.5 nL, about 1 nL, about 3 nL, about 0.01 μL, about 0.1 μL, about 1 μL, about 5 μL, about 10 μL, about 100 μL, about 1 mL, about 5 mL, about 10 mL, or the like. In some cases, the volume of the fluid sample is between about 0.01 μL and about 10 mL, between about 0.01 μL and about 1 mL, between about 0.01 μL and about 100 μL, or between about 0.1 μL and about 10 μL.

In some cases, the fluid sample may be diluted prior to use in an assay described herein. For example, in embodiments the sample is diluted with an appropriate solvent (e.g., a buffer such as PBS buffer). A fluid sample may be diluted about 1-fold, about 2-fold, about 3-fold, about 4-fold, about 5-fold, about 6-fold, about 10-fold, about 100-fold, or greater, prior to use. In other cases, the fluid sample is not diluted prior to use in an assay.

In some cases, the sample may undergo pre-analytical processing. Pre-analytical processing may offer additional functionality such as nonspecific protein removal and/or effective yet cheaply implementable mixing functionality. General methods of pre-analytical processing may include the use of electrokinetic trapping, AC electrokinetics, surface acoustic waves, isotachophoresis, dielectrophoresis, electrophoresis, or other pre-concentration techniques known in the art. In some cases, the fluid sample may be concentrated prior to use in an assay. For example, in embodiments the sample may be concentrated by precipitation, evaporation, filtration, centrifugation, or a combination thereof. A fluid sample may be concentrated about 1-fold, about 2-fold, about 3-fold, about 4-fold, about 5-fold, about 6-fold, about 10-fold, about 100-fold, or greater, prior to use.

It may be desirable to include a calibrator and/or a control in the methods described herein. The calibrator and/or control may be analyzed concurrently with the sample from the subject as described above. The results obtained from the subject sample can be compared to the results obtained from the calibrator and/or control sample. Standard curves may be provided, with which assay results for the sample may be compared. Such standard curves present levels of marker as a function of assay units, i.e., fluorescent signal intensity, if a fluorescent label is used. Using samples taken from multiple donors, standard curves can be provided for reference levels of a target antibody.

### 6. Kits

In some embodiments, the present disclosure further provides kits for performing an immunoassay described herein. In some embodiments, the disclosure further provides kits for detecting antibodies elicited by infection with orthopoxvirus and/or vaccination with vaccinia virus (e.g. vaccination against smallpox) in a sample obtained from a subject. In some embodiments, the kits or systems find use in multiplex and/or automated analysis methods. Exemplary reagents include, but are not limited to, nucleic acid primers, nucleic acid probes, antibodies, colorimetric reagents, enzymes, buffers, etc. Optionally, the kit can also contain at least one calibrator or control. Any calibrator or control can be included in the kit.

In some embodiments, the kit comprises a recombinant A33R protein having at least 80% sequence identity to SEQ ID NO: 1. In some embodiments, the kit comprises a recombinant A35R protein having at least 80% sequence identity to SEQ ID NO: 2. In some embodiments, the kit comprises a recombinant A27L protein having at least 80% sequence identity to SEQ ID NO: 3. In some embodiments, the kit comprises a recombinant B6R protein having at least 80% sequence identity to SEQ ID NO: 4. In some embodiments, the kit comprises a recombinant H3L protein having at least 80% sequence identity to SEQ ID NO: 5. In some embodiments, the kit further comprises a detection moiety. In some embodiments, the detection moiety comprises an anti-human IgG antibody or an anti-human IgM antibody.

Optionally the assays, kits and kit components of the disclosure are optimized for use on commercial platforms (e.g., immunoassays on ARCHITECT® and Alinity) platforms of Abbott Laboratories, Abbott Park, IL, as well as other commercial and/or in vitro diagnostic assays). Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories, Abbott Park, IL) electrochemical immunoassay system and Abbott Rapid Diagnostics Lateral flow tests. Immunosensors and methods of operating them in single-use test devices are described, for example, in U.S. patent applications Ser. No. 20030170881, 20040018577, 20050054078 and 20060160164 which are incorporated herein by reference. Additional background on the manufacture of electrochemical and other types of immunosensors is found in U.S. Pat. No. 5,063,081 which is also incorporated by reference for its teachings regarding same.

Optionally the kits include quality control reagents (for example, sensitivity panels, calibrators, and positive controls). Preparation of quality control reagents is well known in the art, and is described, e.g., on a variety of immunodiagnostic or nucleic acid product insert sheets.

In another embodiment, the present disclosure provides for a quality control kit comprising a capture antigen described herein for use as a sensitivity panel to evaluate assay performance characteristics and/or to quantitate and monitor the integrity of the antigen(s) or antibodies used in the assay.

The kits can optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), may also be included in the kit. The kit may additionally include one or more other controls. One or more of the components of the kit may be lyophilized and the kit may further comprise reagents suitable for the reconstitution of the lyophilized components.

The various components of the kit optionally are provided in suitable containers. As indicated above, one or more of the containers may be a microtiter plate. The kit further can include containers for holding or storing a sample (e.g., a container or cartridge for a blood or urine sample). Where appropriate, the kit may also optionally contain reaction vessels, mixing vessels and other components that facilitate the preparation of reagents or the test sample. The kit may also include one or more instruments for assisting with obtaining a test sample, such as a syringe, pipette, forceps, measured spoon, or the like.

The kit can further optionally include instructions for use, which may be provided in paper form or in computer-readable form, such as a disc, CD, DVD or the like.

The disclosure as described herein also can be adapted for use in a variety of automated and semi-automated systems (including those wherein the solid phase comprises a microparticle), as described, e.g., in U.S. Pat. Nos. 5,089,424 and 5,006,309, and as, e.g., commercially marketed by Abbott Laboratories (Abbott Park, IL) including but not limited to Abbott's ARCHITECT® and Alinity platforms. Moreover, the disclosure optionally is adaptable for the Abbott Laboratories commercial Point of Care (i-STAT™) electrochemical immunoassay system for performing sandwich immunoassays and and Abbott Rapid Diagnostics Lateral flow tests. Immunosensors, and their methods of manufacture and operation in single-use test devices are described, for example in, U.S. Pat. No. 5,063,081, U.S. Patent Application 2003/0170881, U.S. Patent Application 2004/0018577, U.S. Patent Application 2005/0054078, and U.S. Patent Application 2006/0160164, which are incorporated in their entireties by reference for their teachings regarding same.

All patents and publications mentioned in the specification are indicative of the levels of those skilled in the art to which the disclosure pertains. All patents and publications are herein incorporated by reference to the same extent as if each individual publication was specifically and individually indicated to be incorporated by reference.

The disclosure illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed. Thus, it should be understood that although the present disclosure includes various embodiments and optional features, modification and variation of the concepts herein disclosed may be resorted to by those skilled in the art, and that such modifications and variations are considered to be within the scope of this disclosure as defined by the appended claims.

### EXAMPLES

### Example 1

### Assay Development

Using an indirect IgG chemiluminescent microparticle immunoassay (CMIA), several recombinant proteins of Monkeypox and Vaccinia were screened with samples obtained from subject with recent confirmed MPXV infection and two groups of blood donors, one group assumed to have received smallpox vaccine and the other group assumed as having not received vaccines. Results from several recombinant proteins are shown in Table 1.

TABLE 1
Additional screen of recombinant proteins of Monkeypox using IgG CMIA assay

| | | | Indirect Format | | | | | | | | |
|---|---|---|---|---|---|---|---|---|---|---|---|
| | | | Assay File | | | | | | | | |
| | | | 978_193 | | | | | | | | |
| | | | Microparticles | | | | | | | | |
| | | | 25 ug/mL on Styren uP at 0.05% | | | | | | | | |
| | | | Conjugate | | | | | | | | |
| | | | Anti-Human IgG @ 5 ng/mL | | | | | | | | |
| | | | ASD | | | | | | | | |
| | | | Rubella ASD | | | | | | | | |
| | Days | Lot | A35R | A29 | L1R | B6R | H3 | A27L | M1R | I1L | H3L |
| | PSO | Number | (Sino) | (Sino) | (Sino) | (Sino) | (Sino) | (Sino) | (Sino) | (Sino) | (Abbexa) |
| Monkeypox | 6 | 220833 | 138 | 162 | 126 | 696 | 2,069 | 4,265 | 160 | 129 | 522 |
| Infected | 12 | 220835 | 20,272 | 114 | 108 | 39,761 | 32,807 | 48,666 | 209 | 318 | 244 |
| Individuals | 14 | 221028 | 3,210 | 391 | 139 | 3,591 | 9,923 | 39,217 | 170 | 140 | 292 |
| | 24 | 220834 | 9,837 | 490 | 124 | 15,420 | 20,531 | 34,696 | 160 | 151 | 259 |
| | 28 | 221029 | 18,984 | 544 | 111 | 27,916 | 33,175 | 36,353 | 161 | 162 | 259 |
| Blood donors | | 43614 | 167 | 202 | 126 | 685 | 421 | 4,815 | 372 | 331 | 442 |
| (<40 years old) | | 43622 | 299 | 308 | 227 | 499 | 365 | 504 | 387 | 287 | 494 |
| Assumed non- | | 43634 | 134 | 160 | 126 | 1,216 | 460 | 1,670 | 296 | 203 | 596 |
| vaccinees | | 43637 | 172 | 228 | 310 | 459 | 297 | 1,001 | 285 | 235 | 521 |
| | | 43649 | 90 | 148 | 122 | 510 | 383 | 478 | 534 | 350 | 498 |
| | | 43652 | 101 | 131 | 132 | 133 | 226 | 190 | 241 | | |
| | | 43658 | 344 | 348 | 119 | 148 | 389 | 290 | 499 | | |
| Blood donors | | 12263686 | 23,348 | 267 | 190 | 38,341 | 4,301 | 36,426 | 1,543 | 246 | 375 |
| (<40 years old) | | 12263730 | 9,779 | 604 | 398 | 39,504 | 10,322 | 649 | 2,385 | 441 | 654 |
| Assumed | | 12281898 | 36,444 | 475 | 252 | 36,699 | 3,436 | 21,671 | 874 | 471 | 780 |
| vaccinees | | 12281992 | 37,364 | 346 | 254 | 10,236 | 1,809 | 5,752 | 924 | 510 | 785 |
| | | 12312922 | 12,251 | 852 | 151 | 376 | 15,009 | 813 | 1,424 | | |
| | | 12318607 | 21,278 | 360 | 256 | 325 | 20,951 | 533 | 1,167 | | |
| | | 12334472 | 29,711 | 4,481 | 259 | 2,043 | 34,278 | 2,665 | 1,312 | | |

| | | | Indirect Format | | | | | |
|---|---|---|---|---|---|---|---|---|
| | | | Assay File | | | | | |
| | | | 978_193 | | | | | |
| | | | Microparticles | | | | | |
| | | | 25 ug/mL on Styren uP at 0.05% | | | | | |
| | | | Conjugate | | | | | |
| | | | Anti-Human IgG @ 5 ng/mL | | | | | |
| | | | ASD | | | | | |
| | | | Rubella ASD | | | | | |
| | Days | Lot | A26L | A44R | | | | |
| | PSO | Number | (abbexa) | (abbexa) | A29L-AH | A33R-AH | M1R-AH | B21R-AH |
| Monkeypox | 6 | 220833 | 204 | 997 | 298 | 1,000 | 646 | 1.489 |
| Infected | 12 | 220835 | 120 | 3,175 | 1,106 | 37,921 | 423 | 1,389 |
| Individuals | 14 | 221028 | 164 | 1,422 | | 8,799 | | |
| | 24 | 220834 | 136 | 1,074 | 956 | 28,217 | 833 | 1,609 |
| | 28 | 221029 | 124 | 1,242 | | 47,918 | | |
| Blood donors | | 43614 | 293 | 668 | 139 | 304 | 379 | 501 |
| (<40 years old) | | 43622 | 326 | 431 | 274 | 636 | 614 | 944 |
| Assumed non- | | 43634 | 241 | 390 | 99 | 555 | 712 | 1,236 |
| vaccinees | | 43637 | 263 | 561 | 190 | 654 | 431 | 867 |
| | | 43649 | 312 | 598 | 101 | 420 | 425 | 804 |
| | | 43652 | | | | | | |
| | | 43658 | | | | | | |
| Blood donors | | 12263686 | 184 | 1,953 | 199 | 46,863 | 1,008 | 627 |
| (<40 years old) | | 12263730 | 435 | 920 | 375 | 22,882 | 867 | 1,287 |
| Assumed | | 12281898 | 356 | 1,820 | 781 | 48,022 | 526 | 607 |
| vaccinees | | 12281992 | 594 | 1,148 | 290 | 65,642 | 1,249 | 769 |
| | | 12312922 | | | | | | |
| | | 12318607 | | | | | | |
| | | 12334472 | | | | | | |

Compared to other evaluated proteins, A33R, A35R, A27L, B6R and H3L proteins of Monkeypox generated >10-fold higher IgG signal for a majority of the Monkeypox infected human samples and minimal reactivity on the 7 assumed non-vaccines. This data suggests anti-A33R, anti-A35R, anti-A27L, anti-B6R and anti-H3L are the immunodominant responses elicited by Monkeypox infection and may be used as serological markers for detection of OPXV infection/vaccination. The 3 or 7 assumed past vaccines also yielded higher IgG signal with A33R, A35R, A27L, B6R and H3L due to high sequence identity (>86%) between Monkeypox and the Vaccinia virus used for smallpox vaccination. Thus, these markers may be used for detection of the genus Orthopoxvirus infection or detection of smallpox vaccination status.

Using an expressed Monkeypox A33R recombinant protein sequence, (MASILNTLRFLEKTSFYNCNDSITKEKIKIKHKGMSFVFYKPKHSTVVKYLSGGGIYHD DLVVLGKVTINDLKMMLFYMDLSYHGVTSSGAIYKLGSSIDRLSLNRTIVTKVNNNYNN YNNYNNYNCYNNYNCYNYDDTFFDDDDGGGGSHHHHHH) (SEQ ID NO: 6) 4 CMIA assays (IgM, IgG, Total Ig and IgG avidity) were developed. Assay design, key performance and potential utility are summarized below.

Orthopox IgM: An indirect assay format using A33R as capture Ag and anti-human IgM for detection. This assay displayed 100% sensitivity on the 5 Monkeypox samples from Day 6 to 28 post symptom onset and 96% specificity with 111 donors of all ages. The Orthopox IgM assay can be used for screening to identify recent Orthopoxvirus infection/vaccination.

Orthopox IgG: An indirect assay format using A33R as capture Ag and anti-human IgG for detection. This assay displayed 96% sensitivity on the 5 Monkeypox samples and 20 assumed smallpox vaccines and 99% specificity with 98 assumed non-vaccines. The Orthopox IgG assay can be used for screening to identify recent and past Orthopox infection/vaccination.

Orthopox Total Ig: A 1-step direct assay format using A33R as both capture and detection Ag. This assay displayed a sensitivity of 81% on 100 assumed vaccines and 99% specificity with 100 non-vaccines. This assay also displayed 100% detection of 5 Monkeypox samples and 20 assumed Smallpox Vaccines. The prototype can be used for screening to identify recent and past Orthopox infection/vaccination.

Orthopox IgG Avidity: An indirect assay format using A33R as capture & competing Ag and anti-human IgG for detection. All 5 Monkeypox samples had % Avidity <34 vs >53% Avidity from the 16/20 assumed smallpox vaccines. Thus, this assay can be used for reflex testing to differentiate recent vs past Orthopox infection/vaccination.

### Example 2

### Assay Testing

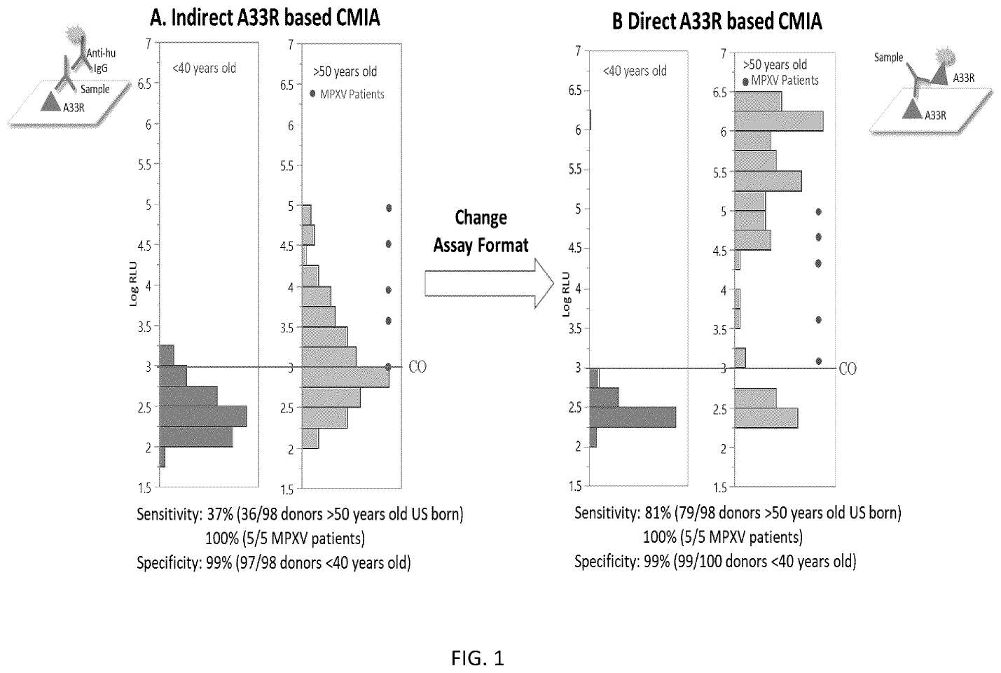

Recombinant A33R protein was further evaluated in indirect and direct assay formats. FIG. 1 shows an evaluation of recombinant A33R protein in (A) indirect anti-human IgG-CMIA and B) direct double antigen Total Ig-CMIA with 98 assumed vaccines (blood donors >50 years old) and 100 assumed non-vaccines (blood donors <40 years old). The sensitivity of the indirect IgG-CMIA (A) using A33R as capture Ag was low (37%) for 98 assumed smallpox vaccines. However, using A33R as both capture and detection Ag in the direct Total Ig-CMIA (B) substantially improved sensitivity from 37% to 81% for the 98 assumed smallpox vaccines while maintaining 99% specificity with 100 assumed non-vaccines, providing further evidence that anti-A33R is one of the immunodominant responses to Orthopoxvirus. Due to high sensitivity and specificity, the A33R based Total Ig-CMIA assay (B) (i.e. Orthopox Total Ig indicated in Example 1) can be used for screening to identify recent and past Orthopox infection/vaccination.

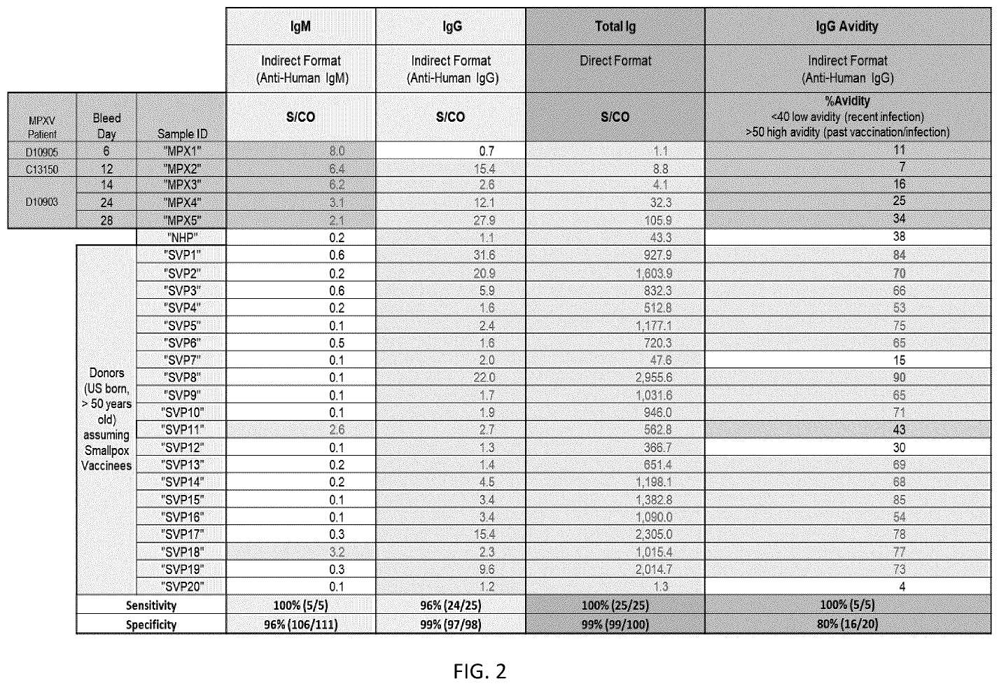

A33R recombinant protein was further evaluated in indirect IgM and IgG, direct Total Ig and indirect IgG Avidity CMIAs. Results are shown in FIG. 2. Results are as follows:

Orthopox IgM: The A33R based IgM-CMIA showed 100% detection of the 5 Monkeypox samples from Day 6 to 28 post symptom onset and 90% specificity with the 20 assumed vaccines, confirming the IgM response to A33R protein of Monkeypox is an early serologic marker and can be used as the aid for diagnosis of acute Monkeypox and other Orthopoxvirus infection.

Orthopox IgG: The A33R based IgG-CMIA showed 96% sensitivity on the 5 Monkeypox samples and 20 assumed vaccines.

Orthopox Total Ig: The A33R based Total Ig-CMIA had 100% sensitivity on the 5 Monkeypox samples and 20 assumed smallpox vaccines. Sensitivity was substantially improved with up to ˜600-fold higher S/CO than the IgG-CMIA, demonstrating anti-A33R response is an immunodominant response and the sensitive prototype can be used for screening to identify recent and past Orthopoxvirus infection/vaccination.

Orthopox IgG Avidity: using A33R as capture & competing Ag in the indirect anti-human IgG format, all 5 Monkeypox samples had % Avidity <34 vs >53% Avidity from the 16/20 assumed smallpox vaccines. The data indicates the IgG avidity prototype can be used as a reflex test to differentiate recent vs past Orthopox infection/vaccination.

For reasons of completeness, various aspects of the disclosure are set out in the following numbered clauses:

Clause 1. A method of detecting a target antibody in a sample, the method comprising:
a) contacting a sample obtained from a subject with a capture antigen and a detection antigen comprising a detectable label, thereby forming a capture antigen-target antibody-detection antigen complex; and
b) detecting a target antibody in the sample based upon a signal generated by the detectable label in the capture antigen-target antibody detection antigen complex, wherein the capture antigen and detection antigen comprise a recombinant A33R, A35R, A27L, B6R, or H3L protein.

Clause 2. The method of clause 1, wherein the capture and/or detection antigen comprise: (i) a recombinant A33R protein having at least 80% sequence identity to SEQ ID NO: 1; (ii) a recombinant A35R protein having at least 80% sequence identity to SEQ ID NO: 2; (iii) a recombinant A27L protein having at least 80% sequence identity to SEQ ID NO: 3; (iv) a recombinant B6R protein having at least 80% sequence identity to SEQ ID NO: 4; or (v) a recombinant H3L protein having at least 80% sequence identity to SEQ ID NO: 5.

Clause 3. The method of clause 1, wherein the capture and/or detection antigen comprise: (i) a recombinant A33R protein having at least 90% sequence identity to SEQ ID NO: 1; (ii) a recombinant A35R protein having at least 90% sequence identity to SEQ ID NO: 2; (iii) a recombinant A27L protein having at least 90% sequence identity to SEQ ID NO: 3; (iv) a recombinant B6R protein having at least 90% sequence identity to SEQ ID NO: 4; or (v) a recombinant H3L protein having at least 90% sequence identity to SEQ ID NO: 5.

Clause 4. The method of clause 1, wherein the capture and/or detection antigen comprise: (i) a recombinant A33R protein having at least 95% sequence identity to SEQ ID NO: 1; (ii) a recombinant A35R protein having at least 95% sequence identity to SEQ ID NO: 2; (iii) a recombinant A27L protein having at least 95% sequence identity to SEQ ID NO: 3; (iv) a recombinant B6R protein having at least 95% sequence identity to SEQ ID NO: 4; or (v) a recombinant H3L protein having at least 95% sequence identity to SEQ ID NO: 5.

Clause 5. The method of clause 1, wherein the capture and/or detection antigen comprise: (i) a recombinant A33R protein having an amino acid sequence of SEQ ID NO: 1; (ii) a recombinant A35R protein having an amino acid sequence of SEQ ID NO: 2; (iii) a recombinant A27L protein having an amino acid sequence of SEQ ID NO: 3; (iv) a recombinant B6R protein having an amino acid sequence of SEQ ID NO: 4; or (v) a recombinant H3L protein having an amino acid sequence of SEQ ID NO: 5.

Clause 6. The method of any one of clauses 1-5, wherein the capture antigen is bound to a solid support and the detection antigen is detectably labeled.

Clause 7. A method of detecting a target antibody in a sample, the method comprising:
a) contacting a sample obtained from a subject with a capture antigen, wherein the capture antigen comprises a recombinant A33R, A35R, A27L, B6R, or H3L protein;
b) contacting the sample with a detection moiety comprising a detectable label, thereby forming a capture antigen-target antibody-detection moiety complex; and
c) detecting a target antibody in the sample based upon a signal generated by the detectable label in the capture antigen-target antibody-detection moiety complex.

Clause 8. The method of clause 7, wherein the capture antigen comprises: (i) a recombinant A33R protein having at least 80% sequence identity to SEQ ID NO: 1; (ii) a recombinant A35R protein having at least 80% sequence identity to SEQ ID NO: 2; (iii) a recombinant A27L protein having at least 80% sequence identity to SEQ ID NO: 3; (iv) a recombinant B6R protein having at least 80% sequence identity to SEQ ID NO: 4; or (v) a recombinant H3L protein having at least 80% sequence identity to SEQ ID NO: 5.

Clause 9. The method of clause 7, wherein the capture antigen comprises: (i) a recombinant A33R protein having at least 90% sequence identity to SEQ ID NO: 1; (ii) a recombinant A35R protein having at least 90% sequence identity to SEQ ID NO: 2; (iii) a recombinant A27L protein having at least 90% sequence identity to SEQ ID NO: 3; (iv) a recombinant B6R protein having at least 90% sequence identity to SEQ ID NO: 4; or (v) a recombinant H3L protein having at least 90% sequence identity to SEQ ID NO: 5.

Clause 10. The method of clause 7, wherein the capture antigen comprises: (i) a recombinant A33R protein having at least 95% sequence identity to SEQ ID NO: 1; (ii) a recombinant A35R protein having at least 95% sequence identity to SEQ ID NO: 2; (iii) a recombinant A27L protein having at least 95% sequence identity to SEQ ID NO: 3; (iv) a recombinant B6R protein having at least 95% sequence identity to SEQ ID NO: 4; or (v) a recombinant H3L protein having at least 95% sequence identity to SEQ ID NO: 5.

Clause 11. The method of clause 7, wherein the capture antigen comprises: (i) a recombinant A33R protein having an amino acid sequence of SEQ ID NO: 1; (ii) a recombinant A35R protein having an amino acid sequence of SEQ ID NO: 2; (iii) a recombinant A27L protein having an amino acid sequence of SEQ ID NO: 3; (iv) a recombinant B6R protein having an amino acid sequence of SEQ ID NO: 4; or (v) a recombinant H3L protein having an amino acid sequence of SEQ ID NO: 5.

Clause 12. The method of any one of clauses 7-11, wherein the capture antigen is bound to a solid support.

Clause 13. The method of any one of clauses 7-12, wherein the detection moiety comprises an anti-human IgG antibody or an anti-human IgM antibody.

Clause 14. The method of any one of clauses 1-13, wherein the subject has or is suspected of having an orthopoxvirus infection.

Clause 15. The method of clause 14, wherein the subject has or is suspected of having a monkeypox infection.

Clause 16. The method of any one of clauses 1-13, wherein the orthopoxvirus vaccination status of the subject is unknown.

Clause 17. The method of any one of clauses 1-13, wherein the subject has received a vaccination against one or more orthopoxviruses.

Clause 18. The method of any one of clauses 1-17, wherein the subject is a human.

Clause 19. The method of any one of clauses 1-18, wherein the sample is a whole blood sample, a serum sample, or a plasma sample.

Clause 20. A method of determining avidity of antibodies in a sample, the method comprising:
a) performing a first assay to determine the total amount of anti-A33R, anti-A35R, anti-A27L, anti-B6R, or anti-H3L antibodies in a sample obtained from a subject; and
b) performing a second assay to determine the amount of low avidity anti-A33R, anti-A35R, anti-A27L, anti-B6R, or anti-H3L antibodies in a sample obtained from the subject.

Clause 21. The method of clause 20, wherein the first assay comprises:
a) contacting a sample obtained from a subject with a solution containing a solid phase antigen such that solid phase antigen-high avidity antibody complexes and solid phase antigen-low avidity antibody complexes are formed in the sample, wherein the solid phase antigen comprises a recombinant A33R, A35R, A27L, B6R or H3L protein attached to a solid phase;
b) adding a detection moiety comprising a detectable label to the sample; and
c) measuring a signal generated by the detectable label in the sample, wherein the signal is indicative of the total amount of anti-A33R, anti-A35R, anti-A27L, anti-B6R, or anti-H3L antibodies present in the sample.

Clause 22. The method of clause 21, wherein the second assay comprises:
a) contacting the sample with a solution containing a solid phase antigen and a liquid phase antigen such that solid phase antigen-low avidity antibody complexes and liquid phase antigen-high avidity antibody complexes are formed in the sample, wherein the solid phase antigen comprises a recombinant A33R, A35R, A27L, B6R or H3L protein attached to a solid phase and the liquid phase antigen comprises a recombinant A33R, A35R, A27L, B6R or H3L protein in solution;
b) washing the sample, thereby removing liquid phase antigen-high avidity antibody complexes from the sample;
c) adding a detection moiety comprising a detectable label to the sample; and
d) measuring a signal generated by the detectable label in the sample, wherein the signal is indicative of the amount of low avidity anti-A33R, anti-A35R, anti-A27L, anti-B6R, or anti-H3L antibodies present in the sample.

Clause 23. The method of clause 22, further comprising determining a ratio of the signal generated by the detectable label in the second assay to the signal generated by the detectable label in the first assay.

Clause 24. The method of clause 23, further comprising determining an avidity index for the subject, wherein the avidity index is determined by subtracting the ratio from 1, then multiplying the ratio by 100, wherein the avidity index is indicative of the amount of high-avidity anti-A33R anti-A35R, anti-A27L, anti-B6R, or anti-H3L antibodies in a sample obtained from the subject.

Clause 25. The method of clause 24, wherein an avidity index of less than 45% indicates that the subject is recently infected or vaccinated with an orthopoxvirus, and wherein an avidity index of greater than 45% indicates that the subject has previously received a vaccination against one or more orthopoxviruses.

Clause 26. The method of any one of clauses 20-25, wherein the sample is a whole blood sample, a serum sample, or a plasma sample.

Clause 27. The method of any one of clauses 20-26, wherein the subject is a human.

Clause 28. A method of differentiating between an orthopoxvirus infection and previous orthopoxvirus vaccination in a subject, the method comprising:
a) performing a first assay to determine the total amount of anti-A33R, anti-A35R, anti-A27L, anti-B6R, or anti-H3L antibodies in a sample obtained from a subject;
b) performing a second assay to determine the amount of low avidity anti-A33R, anti-A35R, anti-A27L, anti-B6R, or anti-H3L antibodies in a sample obtained from the subject; and
c) differentiating between an orthopoxvirus infection and previous orthopoxvirus vaccination in the subject based upon a ratio of the amount of low avidity anti-A33R, anti-A35R, anti-A27L, anti-B6R, or anti-H3L antibodies to the amount of total anti-A33R anti-A35R, anti-A27L, anti-B6R, or anti-H3L antibodies determined in the first and second assays.

Clause 29. The method of clause 28, wherein the first assay comprises:
a) contacting a sample obtained from a subject with a solution containing a solid phase antigen such that solid phase antigen-high avidity antibody complexes and solid phase antigen-low avidity antibody complexes are formed in the sample, wherein the solid phase antigen comprises a recombinant A33R, A35R, A27L, B6R or H3L protein attached to a solid phase;
b) adding a detection moiety comprising a detectable label to the sample; and
c) measuring a signal generated by the detectable label in the sample, wherein the signal is indicative of the total amount of anti-A33R anti-A35R, anti-A27L, anti-B6R, or anti-H3L antibodies present in the sample.

Clause 30. The method of clause 29, wherein the second assay comprises:
a) contacting the sample with a solution containing a solid phase antigen and a liquid phase antigen such that solid phase antigen-low avidity antibody complexes and liquid phase antigen-high avidity antibody complexes are formed in the sample, wherein the solid phase antigen comprises a recombinant A33R, A35R, A27L, B6R or H3L protein attached to a solid phase and the liquid phase antigen comprises a recombinant A33R, A35R, A27L, B6R or H3L protein in solution;
b) washing the sample, thereby removing liquid phase antigen-high avidity antibody complexes from the sample;
c) adding a detection moiety comprising a detectable label to the sample; and
d) measuring a signal generated by the detectable label in the sample, wherein the signal is indicative of the amount of low avidity anti-A33R, anti-A35R, anti-A27L, anti-B6R, or anti-H3L antibodies present in the sample.

Clause 31. The method of clause 30, further comprising determining a ratio of the signal generated by the detectable label in the second assay to the signal generated by the detectable label in the first assay.

Clause 32. The method of clause 31, further comprising determining an avidity index for the subject, wherein the avidity index is determined by subtracting the ratio from 1, then multiplying the ratio by 100, wherein the avidity index is indicative of the amount of high-avidity anti-A33R, anti-A35R, anti-A27L, anti-B6R, or anti-H3L antibodies in a sample obtained from the subject.

Clause 33. The method of clause 32, comprising determining that the subject is recently infected or vaccinated with an orthopoxvirus when the avidity index is less than 45%, or determining that the subject has previously received a vaccination against one or more orthopoxviruses when the avidity index of is greater than 45%.

Clause 34. The method of any one of clauses 28-33, wherein the sample is a whole blood sample, a serum sample, or a plasma sample.

Clause 35. The method of any one of clauses 28-34, wherein the subject is a human.

Clause 35. A system comprising:
a sample receiving component configured to receive a sample from a subject;
a capture antigen and detection antigen comprising a detectable label configured to make contact with the sample to form a capture antigen-target antibody-detection antigen complex, wherein the capture/detection antigen comprises a recombinant A33R, A35R, A27L, B6R or H3L protein;
a detection component configured to measure a signal generated by the detectable label in the capture antigen-target antibody-detection antigen complex; and
an output component that indicates an amount of target antibody in the sample based on the signal.

Clause 36. The system of clause 35, wherein the capture and/or detection antigen comprise: (i) a recombinant A33R protein having at least 80% sequence identity to SEQ ID NO: 1; (ii) a recombinant A35R protein having at least 80% sequence identity to SEQ ID NO: 2; (iii) a recombinant A27L protein having at least 80% sequence identity to SEQ ID NO: 3; (iv) a recombinant B6R protein having at least 80% sequence identity to SEQ ID NO: 4; or (v) a recombinant H3L protein having at least 80% sequence identity to SEQ ID NO: 5.

Clause 37. The system of clause 36, wherein the capture and/or detection antigen comprise: (i) a recombinant A33R protein having at least 90% sequence identity to SEQ ID NO: 1; (ii) a recombinant A35R protein having at least 90% sequence identity to SEQ ID NO: 2; (iii) a recombinant A27L protein having at least 90% sequence identity to SEQ ID NO: 3; (iv) a recombinant B6R protein having at least 90% sequence identity to SEQ ID NO: 4; or (v) a recombinant H3L protein having at least 90% sequence identity to SEQ ID NO: 5.

Clause 38. The system of clause 36, wherein the capture and/or detection antigen comprise: (i) a recombinant A33R protein having at least 95% sequence identity to SEQ ID NO: 1; (ii) a recombinant A35R protein having at least 95% sequence identity to SEQ ID NO: 2; (iii) a recombinant A27L protein having at least 95% sequence identity to SEQ ID NO: 3; (iv) a recombinant B6R protein having at least 95% sequence identity to SEQ ID NO: 4; or (v) a recombinant H3L protein having at least 95% sequence identity to SEQ ID NO: 5.

Clause 39. The system of clause 36, wherein the capture and/or detection antigen comprise: (i) a recombinant A33R protein having an amino acid sequence of SEQ ID NO: 1; (ii) a recombinant A35R protein having an amino acid sequence of SEQ ID NO: 2; (iii) a recombinant A27L protein having an amino acid sequence of SEQ ID NO: 3; (iv) a recombinant B6R protein having an amino acid sequence of SEQ ID NO: 4; or (v) a recombinant H3L protein having an amino acid sequence of SEQ ID NO: 5.

Clause 40. A system of detecting a target antibody in a sample, the system comprising:
a) contacting a sample obtained from a subject with a capture antigen, wherein the capture antigen comprises a recombinant A33R, A35R, A27L, B6R or H3L protein;
b) contacting the sample with a detection moiety comprising a detectable label, thereby forming a capture antigen-target antibody-detection moiety complex; and
c) detecting a target antibody in the sample based upon a signal generated by the detectable label in the capture antigen-target antibody-detection moiety complex.

Clause 41. The system of clause 41, wherein the capture antigen comprises: (i) a recombinant A33R protein having at least 80% sequence identity to SEQ ID NO: 1; (ii) a recombinant A35R protein having at least 80% sequence identity to SEQ ID NO: 2; (iii) a recombinant A27L protein having at least 80% sequence identity to SEQ ID NO: 3; (iv) a recombinant B6R protein having at least 90% sequence identity to SEQ ID NO: 4; or (v) a recombinant H3L protein having at least 80% sequence identity to SEQ ID NO: 5.

Clause 42. The system of clause 41, wherein the capture antigen comprises: (i) a recombinant A33R protein having at least 90% sequence identity to SEQ ID NO: 1; (ii) a recombinant A35R protein having at least 90% sequence identity to SEQ ID NO: 2; (iii) a recombinant A27L protein having at least 90% sequence identity to SEQ ID NO: 3; (iv) a recombinant B6R protein having at least 90% sequence identity to SEQ ID NO: 4; or (v) a recombinant H3L protein having at least 90% sequence identity to SEQ ID NO: 5.

Clause 43. The system of clause 41, wherein the capture antigen comprises: (i) a recombinant A33R protein having at least 95% sequence identity to SEQ ID NO: 1; (ii) a recombinant A35R protein having at least 95% sequence identity to SEQ ID NO: 2; (iii) a recombinant A27L protein having at least 95% sequence identity to SEQ ID NO: 3; (iv) a recombinant B6R protein having at least 95% sequence identity to SEQ ID NO: 4; or (v) a recombinant H3L protein having at least 95% sequence identity to SEQ ID NO: 5.

Clause 44. The system of clause 41, wherein the capture antigen comprises: (i) a recombinant A33R protein having an amino acid sequence of SEQ ID NO: 1; (ii) a recombinant A35R protein having an amino acid sequence of SEQ ID NO: 2; (iii) a recombinant A27L protein having an amino acid sequence of SEQ ID NO: 3; (iv) a recombinant B6R protein having an amino acid sequence of SEQ ID NO: 4; or (v) a recombinant H3L protein having an amino acid sequence of SEQ ID NO: 5.

Clause 45. The system of any one of clauses 41-44, wherein the detection moiety comprises an anti-human IgG antibody or an anti-human IgM antibody.

Clause 46. The system of any one of clauses 41-45, wherein the subject has or is suspected of having an orthopoxvirus infection.

Clause 47. The system of clause 46, wherein the subject has or is suspected of having a monkeypox infection.

Clause 48. The system of any one of clauses 41-47, wherein the orthopoxvirus vaccination status of the subject is unknown.

Clause 49. The system of any one of clauses 41-48, wherein the subject has received a vaccination against one or more orthopoxviruses.

Clause 50. The system of any one of clauses 41-49, wherein the subject is a human.

Clause 51. The system of any one of clauses 41-50, wherein the sample is a whole blood sample, a serum sample, or a plasma sample.

Clause 52. The system of any one of clauses 41-51, wherein the capture antigen is bound to a solid support.

Clause 53. A system comprising:
a sample receiving component configured to receive a sample from a subject;
a sample analysis component configured to determine a total amount of anti-A33R antibodies in the sample and an amount of low avidity anti-A33R, anti-A35R, anti-A27L, anti-B6R, or anti-H3L antibodies in the sample to arrive at an index representing a relationship between the total amount of anti-A33R, anti-A35R, anti-A27L, anti-B6R, or anti-H3L antibodies and the amount of low avidity anti-A33R, anti-A35R, anti-A27L, anti-B6R, or anti-H3L antibodies; and
an output component that indicates 1) the total amount of anti-A33R, anti-A35R, anti-A27L, anti-B6R, or anti-H3L antibodies and the amount of low avidity anti-A33R, anti-A35R, anti-A27L, anti-B6R, or anti-H3L antibodies, 2) the index, 3) whether the index is above or below a preset threshold, and/or 4) whether the subject is likely infected with an orthopoxvirus or has received a vaccination against one or more orthopoxviruses.

Clause 54. The system of clause 53, wherein the index is determined by subtracting the ratio from 1, then multiplying the ratio by 100, wherein the avidity index is indicative of the amount of high-avidity anti-A33R, anti-A35R, anti-A27L, anti-B6R, or anti-H3L antibodies in a sample obtained from the subject.

Clause 55. The system of clause 53 or clause 54, wherein an index of less than 45% indicates that the subject is recently infected or vaccinated with an orthopoxvirus, and wherein an index of greater than 45% indicates that the subject has previously received a vaccination against one or more orthopoxviruses.

Clause 56. The system of any one of clauses 53-55, wherein the sample is a whole blood sample, a serum sample, or a plasma sample.

Clause 57. The system of any one of clauses 53-56, wherein the subject is a human.

What is claimed is:

1. A method of detecting a target antibody in a sample, the method comprising:
a) contacting a sample obtained from a subject with a capture antigen and a detection antigen comprising a detectable label, thereby forming a capture antigen-target antibody-detection antigen complex; and
b) detecting a target antibody in the sample based upon a signal generated by the detectable label in the capture antigen-target antibody detection antigen complex, wherein the capture antigen and detection antigen comprise a recombinant A33R, A35R, A27L, B6R, or H3L protein.

2. The method of claim 1, wherein the capture and/or detection antigen comprise: (i) a recombinant A33R protein having at least 80% sequence identity to SEQ ID NO: 1; (ii) a recombinant A35R protein having at least 80% sequence identity to SEQ ID NO: 2; (iii) a recombinant A27L protein having at least 80% sequence identity to SEQ ID NO: 3; (iv) a recombinant B6R protein having at least 80% sequence identity to SEQ ID NO: 4; or (v) a recombinant H3L protein having at least 80% sequence identity to SEQ ID NO: 5.

3. The method of claim 1, wherein the capture and/or detection antigen comprise: (i) a recombinant A33R protein having at least 90% sequence identity to SEQ ID NO: 1; (ii) a recombinant A35R protein having at least 90% sequence identity to SEQ ID NO: 2; (iii) a recombinant A27L protein having at least 90% sequence identity to SEQ ID NO: 3; (iv) a recombinant B6R protein having at least 90% sequence identity to SEQ ID NO: 4; or (v) a recombinant H3L protein having at least 90% sequence identity to SEQ ID NO: 5.

4. The method of claim 1, wherein the capture and/or detection antigen comprise: (i) a recombinant A33R protein having at least 95% sequence identity to SEQ ID NO: 1; (ii) a recombinant A35R protein having at least 95% sequence identity to SEQ ID NO: 2; (iii) a recombinant A27L protein having at least 95% sequence identity to SEQ ID NO: 3; (iv) a recombinant B6R protein having at least 95% sequence identity to SEQ ID NO: 4; or (v) a recombinant H3L protein having at least 95% sequence identity to SEQ ID NO: 5.

5. The method of claim 1, wherein the capture and/or detection antigen comprise: (i) a recombinant A33R protein having an amino acid sequence of SEQ ID NO: 1; (ii) a recombinant A35R protein having an amino acid sequence of SEQ ID NO: 2; (iii) a recombinant A27L protein having an amino acid sequence of SEQ ID NO: 3; (iv) a recombinant B6R protein having an amino acid sequence of SEQ ID NO: 4; or (v) a recombinant H3L protein having an amino acid sequence of SEQ ID NO: 5.

6. The method of any one of claims 1-5, wherein the capture antigen is bound to a solid support and the detection antigen is detectably labeled.

7. A method of detecting a target antibody in a sample, the method comprising:
a) contacting a sample obtained from a subject with a capture antigen, wherein the capture antigen comprises a recombinant A33R, A35R, A27L, B6R, or H3L protein;
b) contacting the sample with a detection moiety comprising a detectable label, thereby forming a capture antigen-target antibody-detection moiety complex; and
c) detecting a target antibody in the sample based upon a signal generated by the detectable label in the capture antigen-target antibody-detection moiety complex.

8. The method of claim 7, wherein the capture antigen comprises: (i) a recombinant A33R protein having at least 80% sequence identity to SEQ ID NO: 1; (ii) a recombinant A35R protein having at least 80% sequence identity to SEQ ID NO: 2; (iii) a recombinant A27L protein having at least 80% sequence identity to SEQ ID NO: 3; (iv) a recombinant B6R protein having at least 80% sequence identity to SEQ ID NO: 4; or (v) a recombinant H3L protein having at least 80% sequence identity to SEQ ID NO: 5.

9. The method of claim 7, wherein the capture antigen comprises: (i) a recombinant A33R protein having at least 90% sequence identity to SEQ ID NO: 1; (ii) a recombinant A35R protein having at least 90% sequence identity to SEQ ID NO: 2; (iii) a recombinant A27L protein having at least 90% sequence identity to SEQ ID NO: 3; (iv) a recombinant B6R protein having at least 90% sequence identity to SEQ ID NO: 4; or (v) a recombinant H3L protein having at least 90% sequence identity to SEQ ID NO: 5

10. The method of claim 7, wherein the capture antigen comprises: (i) a recombinant A33R protein having at least 95% sequence identity to SEQ ID NO: 1; (ii) a recombinant A35R protein having at least 95% sequence identity to SEQ ID NO: 2; (iii) a recombinant A27L protein having at least 95% sequence identity to SEQ ID NO: 3; (iv) a recombinant B6R protein having at least 95% sequence identity to SEQ ID NO: 4; or (v) a recombinant H3L protein having at least 95% sequence identity to SEQ ID NO: 5

11. The method of claim 7, wherein the capture antigen comprises: (i) a recombinant A33R protein having an amino acid sequence of SEQ ID NO: 1; (ii) a recombinant A35R protein having an amino acid sequence of SEQ ID NO: 2; (iii) a recombinant A27L protein having an amino acid sequence of SEQ ID NO: 3; (iv) a recombinant B6R protein having an amino acid sequence of SEQ ID NO: 4; or (v) a recombinant H3L protein having an amino acid sequence of SEQ ID NO: 5.

12. The method of any one of claims 7-11, wherein the capture antigen is bound to a solid support.

13. The method of any one of claims 7-12, wherein the detection moiety comprises an anti-human IgG antibody or an anti-human IgM antibody.

14. The method of any one of claims 1-13, wherein the subject has or is suspected of having an orthopoxvirus infection.

15. The method of claim 14, wherein the subject has or is suspected of having a monkeypox infection.

16. The method of any one of claims 1-13, wherein the orthopoxvirus vaccination status of the subject is unknown.

17. The method of any one of claims 1-13, wherein the subject has received a vaccination against one or more orthopoxviruses.

18. The method of any one of claims 1-17, wherein the subject is a human.

19. The method of any one of claims 1-18, wherein the sample is a whole blood sample, a serum sample, or a plasma sample.

20. A method of determining avidity of antibodies in a sample, the method comprising:
a) performing a first assay to determine the total amount of anti-A33R, anti-A35R, anti-A27L, anti-B6R, or anti-H3L antibodies in a sample obtained from a subject; and
b) performing a second assay to determine the amount of low avidity anti-A33R, anti-A35R, anti-A27L, anti-B6R, or anti-H3L antibodies in a sample obtained from the subject.

21. The method of claim 20, wherein the first assay comprises:
a) contacting a sample obtained from a subject with a solution containing a solid phase antigen such that solid phase antigen-high avidity antibody complexes and solid phase antigen-low avidity antibody complexes are formed in the sample, wherein the solid phase antigen comprises a recombinant A33R, A35R, A27L, B6R or H3L protein attached to a solid phase;
b) adding a detection moiety comprising a detectable label to the sample; and
c) measuring a signal generated by the detectable label in the sample, wherein the signal is indicative of the total amount of anti-A33R, anti-A35R, anti-A27L, anti-B6R, or anti-H3L antibodies present in the sample.

22. The method of claim 21, wherein the second assay comprises:
a) contacting the sample with a solution containing a solid phase antigen and a liquid phase antigen such that solid phase antigen-low avidity antibody complexes and liquid phase antigen-high avidity antibody complexes are formed in the sample, wherein the solid phase antigen comprises a recombinant A33R, A35R, A27L, B6R or H3L protein attached to a solid phase and the liquid phase antigen comprises a recombinant A33R, A35R, A27L, B6R or H3L protein in solution;
b) washing the sample, thereby removing liquid phase antigen-high avidity antibody complexes from the sample;
c) adding a detection moiety comprising a detectable label to the sample; and
d) measuring a signal generated by the detectable label in the sample, wherein the signal is indicative of the amount of low avidity anti-A33R, anti-A35R, anti-A27L, anti-B6R, or anti-H3L antibodies present in the sample.

23. The method of claim 22, further comprising determining a ratio of the signal generated by the detectable label in the second assay to the signal generated by the detectable label in the first assay.

24. The method of claim 23, further comprising determining an avidity index for the subject, wherein the avidity index is determined by subtracting the ratio from 1, then multiplying the ratio by 100, wherein the avidity index is indicative of the amount of high-avidity anti-A33R anti-A35R, anti-A27L, anti-B6R, or anti-H3L antibodies in a sample obtained from the subject.

25. The method of claim 24, wherein an avidity index of less than 45% indicates that the subject is recently infected or vaccinated with an orthopoxvirus, and wherein an avidity index of greater than 45% indicates that the subject has previously received a vaccination against one or more orthopoxviruses.

26. The method of any one of claims 20-25, wherein the sample is a whole blood sample, a serum sample, or a plasma sample.

27. The method of any one of claims 20-26, wherein the subject is a human.

28. A method of differentiating between an orthopoxvirus infection and previous orthopoxvirus vaccination in a subject, the method comprising:
a) performing a first assay to determine the total amount of anti-A33R, anti-A35R, anti-A27L, anti-B6R, or anti-H3L antibodies in a sample obtained from a subject;
b) performing a second assay to determine the amount of low avidity anti-A33R, anti-A35R, anti-A27L, anti-B6R, or anti-H3L antibodies in a sample obtained from the subject; and
c) differentiating between an orthopoxvirus infection and previous orthopoxvirus vaccination in the subject based upon a ratio of the amount of low avidity anti-A33R, anti-A35R, anti-A27L, anti-B6R, or anti-H3L antibodies to the amount of total anti-A33R anti-A35R, anti-A27L, anti-B6R, or anti-H3L antibodies determined in the first and second assays.

29. The method of claim 28, wherein the first assay comprises:
a) contacting a sample obtained from a subject with a solution containing a solid phase antigen such that solid phase antigen-high avidity antibody complexes and solid phase antigen-low avidity antibody complexes are formed in the sample, wherein the solid phase antigen comprises a recombinant A33R, A35R, A27L, B6R or H3L protein attached to a solid phase;
b) adding a detection moiety comprising a detectable label to the sample; and
c) measuring a signal generated by the detectable label in the sample, wherein the signal is indicative of the total amount of anti-A33R anti-A35R, anti-A27L, anti-B6R, or anti-H3L antibodies present in the sample.

30. The method of claim 29, wherein the second assay comprises:
a) contacting the sample with a solution containing a solid phase antigen and a liquid phase antigen such that solid phase antigen-low avidity antibody complexes and liquid phase antigen-high avidity antibody complexes are formed in the sample, wherein the solid phase antigen comprises a recombinant A33R, A35R, A27L, B6R or H3L protein attached to a solid phase and the liquid phase antigen comprises a recombinant A33R, A35R, A27L, B6R or H3L protein in solution;
b) washing the sample, thereby removing liquid phase antigen-high avidity antibody complexes from the sample;
c) adding a detection moiety comprising a detectable label to the sample; and
d) measuring a signal generated by the detectable label in the sample, wherein the signal is indicative of the amount of low avidity anti-A33R, anti-A35R, anti-A27L, anti-B6R, or anti-H3L antibodies present in the sample.

31. The method of claim 30, further comprising determining a ratio of the signal generated by the detectable label in the second assay to the signal generated by the detectable label in the first assay.

32. The method of claim 31, further comprising determining an avidity index for the subject, wherein the avidity index is determined by subtracting the ratio from 1, then multiplying the ratio by 100, wherein the avidity index is indicative of the amount of high-avidity anti-A33R, anti-A35R, anti-A27L, anti-B6R, or anti-H3L antibodies in a sample obtained from the subject.

33. The method of claim 32, comprising determining that the subject is recently infected or vaccinated with an orthopoxvirus when the avidity index is less than 45%, or determining that the subject has previously received a vaccination against one or more orthopoxviruses when the avidity index of is greater than 45%.

34. The method of any one of claims 28-33, wherein the sample is a whole blood sample, a serum sample, or a plasma sample.

35. The method of any one of claims 28-34, wherein the subject is a human.

36. A system comprising:
a sample receiving component configured to receive a sample from a subject;
a capture antigen and detection antigen comprising a detectable label configured to make contact with the sample to form a capture antigen-target antibody-detection antigen complex, wherein the capture/detection antigen comprises a recombinant A33R, A35R, A27L, B6R or H3L protein;
a detection component configured to measure a signal generated by the detectable label in the capture antigen-target antibody-detection antigen complex; and
an output component that indicates an amount of target antibody in the sample based on the signal.

37. The system of claim 36, wherein the capture and/or detection antigen comprise: (i) a recombinant A33R protein having at least 80% sequence identity to SEQ ID NO: 1; (ii) a recombinant A35R protein having at least 80% sequence identity to SEQ ID NO: 2; (iii) a recombinant A27L protein having at least 80% sequence identity to SEQ ID NO: 3; (iv) a recombinant B6R protein having at least 80% sequence identity to SEQ ID NO: 4; or (v) a recombinant H3L protein having at least 80% sequence identity to SEQ ID NO: 5

38. The system of claim 36, wherein the capture and/or detection antigen comprise: (i) a recombinant A33R protein having at least 90% sequence identity to SEQ ID NO: 1; (ii) a recombinant A35R protein having at least 90% sequence identity to SEQ ID NO: 2; (iii) a recombinant A27L protein having at least 90% sequence identity to SEQ ID NO: 3; (iv) a recombinant B6R protein having at least 90% sequence identity to SEQ ID NO: 4; or (v) a recombinant H3L protein having at least 90% sequence identity to SEQ ID NO: 5

39. The system of claim 36, wherein the capture and/or detection antigen comprise: (i) a recombinant A33R protein having at least 95% sequence identity to SEQ ID NO: 1; (ii) a recombinant A35R protein having at least 95% sequence identity to SEQ ID NO: 2; (iii) a recombinant A27L protein having at least 95% sequence identity to SEQ ID NO: 3; (iv) a recombinant B6R protein having at least 95% sequence identity to SEQ ID NO: 4; or (v) a recombinant H3L protein having at least 95% sequence identity to SEQ ID NO: 5.

40. The system of claim 36, wherein the capture and/or detection antigen comprise: (i) a recombinant A33R protein having an amino acid sequence of SEQ ID NO: 1; (ii) a recombinant A35R protein having an amino acid sequence of SEQ ID NO: 2; (iii) a recombinant A27L protein having an amino acid sequence of SEQ ID NO: 3; (iv) a recombinant B6R protein having an amino acid sequence of SEQ ID NO: 4; or (v) a recombinant H3L protein having an amino acid sequence of SEQ ID NO: 5.

41. A system of detecting a target antibody in a sample, the system comprising:
a) contacting a sample obtained from a subject with a capture antigen, wherein the capture antigen comprises a recombinant A33R, A35R, A27L, B6R or H3L protein;
b) contacting the sample with a detection moiety comprising a detectable label, thereby forming a capture antigen-target antibody-detection moiety complex; and
c) detecting a target antibody in the sample based upon a signal generated by the detectable label in the capture antigen-target antibody-detection moiety complex.

42. The system of claim 41, wherein the capture antigen comprises: (i) a recombinant A33R protein having at least 80% sequence identity to SEQ ID NO: 1; (ii) a recombinant A35R protein having at least 80% sequence identity to SEQ ID NO: 2; (iii) a recombinant A27L protein having at least 80% sequence identity to SEQ ID NO: 3; (iv) a recombinant B6R protein having at least 90% sequence identity to SEQ ID NO: 4; or (v) a recombinant H3L protein having at least 80% sequence identity to SEQ ID NO: 5.

43. The system of claim 41, wherein the capture antigen comprises: (i) a recombinant A33R protein having at least 90% sequence identity to SEQ ID NO: 1; (ii) a recombinant A35R protein having at least 90% sequence identity to SEQ ID NO: 2; (iii) a recombinant A27L protein having at least 90% sequence identity to SEQ ID NO: 3; (iv) a recombinant B6R protein having at least 90% sequence identity to SEQ ID NO: 4; or (v) a recombinant H3L protein having at least 90% sequence identity to SEQ ID NO: 5.

44. The system of claim 41, wherein the capture antigen comprises: (i) a recombinant A33R protein having at least 95% sequence identity to SEQ ID NO: 1; (ii) a recombinant A35R protein having at least 95% sequence identity to SEQ ID NO: 2; (iii) a recombinant A27L protein having at least 95% sequence identity to SEQ ID NO: 3; (iv) a recombinant B6R protein having at least 95% sequence identity to SEQ ID NO: 4; or (v) a recombinant H3L protein having at least 95% sequence identity to SEQ ID NO: 5.

45. The system of claim 41, wherein the capture antigen comprises: (i) a recombinant A33R protein having an amino acid sequence of SEQ ID NO: 1; (ii) a recombinant A35R protein having an amino acid sequence of SEQ ID NO: 2; (iii) a recombinant A27L protein having an amino acid sequence of SEQ ID NO: 3; (iv) a recombinant B6R protein having an amino acid sequence of SEQ ID NO: 4; or (v) a recombinant H3L protein having an amino acid sequence of SEQ ID NO: 5.

46. The system of any one of claims 41-45, wherein the detection moiety comprises an anti-human IgG antibody or an anti-human IgM antibody.

47. The system of any one of claims 41-46, wherein the subject has or is suspected of having an orthopoxvirus infection.

48. The system of claim 47, wherein the subject has or is suspected of having a monkeypox infection.

49. The system of any one of claims 41-48, wherein the orthopoxvirus vaccination status of the subject is unknown.

50. The system of any one of claims 41-49, wherein the subject has received a vaccination against one or more orthopoxviruses.

51. The system of any one of claims 41-50, wherein the subject is a human.

52. The system of any one of claims 41-51, wherein the sample is a whole blood sample, a serum sample, or a plasma sample.

53. The system of any one of claims 41-52, wherein the capture antigen is bound to a solid support.

54. A system comprising:
a sample receiving component configured to receive a sample from a subject;
a sample analysis component configured to determine a total amount of anti-A33R antibodies in the sample and an amount of low avidity anti-A33R, anti-A35R, anti-A27L, anti-B6R, or anti-H3L antibodies in the sample to arrive at an index representing a relationship between the total amount of anti-A33R, anti-A35R, anti-A27L, anti-B6R, or anti-H3L antibodies and the amount of low avidity anti-A33R, anti-A35R, anti-A27L, anti-B6R, or anti-H3L antibodies; and
an output component that indicates 1) the total amount of anti-A33R, anti-A35R, anti-A27L, anti-B6R, or anti-H3L antibodies and the amount of low avidity anti-A33R, anti-A35R, anti-A27L, anti-B6R, or anti-H3L antibodies, 2) the index, 3) whether the index is above or below a preset threshold, and/or 4) whether the subject is likely infected with an orthopoxvirus or has received a vaccination against one or more orthopoxviruses.

55. The system of claim 54, wherein the index is determined by subtracting the ratio from 1, then multiplying the ratio by 100, wherein the avidity index is indicative of the amount of high-avidity anti-A33R, anti-A35R, anti-A27L, anti-B6R, or anti-H3L antibodies in a sample obtained from the subject.

56. The system of claim 54 or claim 55, wherein an index of less than 45% indicates that the subject is recently infected or vaccinated with an orthopoxvirus, and wherein an index of greater than 45% indicates that the subject has previously received a vaccination against one or more orthopoxviruses.

57. The system of any one of claims 54-56, wherein the sample is a whole blood sample, a serum sample, or a plasma sample.

58. The system of any one of claims 54-57, wherein the subject is a human.
